(19) United States
(12) Patent Application Publication
(10) Pub. No.: US 2014/0330143 A1
(43) Pub. Date: Nov. 6, 2014

(54) METHOD AND SYSTEM FOR TREATING CARDIOVASCULAR DISEASE

(71) Applicant: CARDIOMEMS, INC., Atlanta, GA (US)

(72) Inventors: JASON KROH, Atlanta, GA (US); ERIK MOORE, Roswell, GA (US); TAMARA SCHOLZ, Atlanta, GA (US); ADRIANE DUREY, SMYRNA, GA (US); JASON WHITE, Decatur, GA (US); KHIN KHIN LAY-KHAN, Atlanta, GA (US)

(73) Assignee: CARDIOMEMS, INC., Atlanta, GA (US)

(21) Appl. No.: 14/268,886

(22) Filed: May 2, 2014

Related U.S. Application Data
(60) Provisional application No. 61/820,510, filed on May 7, 2013.
(60) Provisional application No. 61/819,152, filed on May 3, 2013.

(51) Int. Cl. A61B 5/0215

(52) U.S. Cl. CPC A61B 5/0215
USPC 600/486

(57) ABSTRACT

A system and method for treating congestive heart failure in a patient, including: implanting at least one pressure sensor in a desired location within the patient; providing an ex-vivo interrogation system and monitoring system that can be configured to optionally affect at least one of: selectively energizing the at one pressure sensor, receiving a return or output signal from the at one pressure sensor, processing the return signal, and displaying processed data derived from the at least one pressure sensor to a physician. The system and method also includes deriving diagnostic and treatment information from the processed data and sending diagnostic and treatment information to the patient.

### CROSS REFERENCE TO RELATED APPLICATIONS

This application claims the benefit of U.S. Provisional Application No. 61/820,510 filed May 7, 2013 and U.S. Application No. 61/819,152 filed May 3, 2013, which is hereby incorporated herein by references in its entirety.

### BACKGROUND OF THE INVENTION

1. Field of the Invention

This invention relates generally to systems and methods for detecting and diagnosing cardiovascular disease in a medical patient

2. Description of the Related Art

The optimum management of patients with chronic diseases requires that therapy be adjusted in response to changes in the patient's condition. Ideally, these changes are measured by daily patient self-monitoring prior to the development of symptoms. Self-monitoring and self-administration of therapy forms a closed therapeutic loop, creating a dynamic management system for maintaining homeostasis. Such a system can, in the short term, benefit day-to-day symptoms and quality-of-life, and in the long term, prevent progressive deterioration and complications.

In some cases, timely administration of a single dose of a therapy can prevent serious acute changes in the patient's condition. In another cases, interactive physician management strategies can impact both the short and long term sequalae of the illness. For example, a patient can correspondingly adjust their medications according to their physician's prescription and the prescription can be adjusted by the physician based on changes in the patient's underlying condition. With continual monitoring of the subject physiological parameter(s) of interest, changes in disease management can be made by the physician in order to prevent hospitalization due to symptoms caused by under-treatment and over-treatment.

There are approximately 5 million patients with symptoms relating to underlying heart damage defining a clinical condition known as congestive heart failure (CHF). Although survival rates have improved, the mortality associated with CHF remains worse than many common cancers. The number of CHF patients is expected to grow to 10 million within the coming decade as the population ages and more people with damaged hearts are surviving. CHF is a condition in which a patient's heart works less efficiently than it should, and a condition in which the heart fails to supply the body sufficiently with the oxygen-rich blood it requires, either during exercise or at rest. To compensate for this condition and to maintain blood flow (cardiac output), the body retains sodium and water such that there is a build-up of fluid hydrostatic pressure in the pulmonary blood vessels that drain the lungs. As this hydrostatic pressure overwhelms oncotic pressure and lymph flow, fluid transudates from the pulmonary veins into the pulmonary interstitial spaces, and eventually into the alveolar air spaces. This complication of CHF is called pulmonary edema, which can cause shortness of breath, hypoxemia, acidosis, respiratory arrest, and death. Although CHF is a chronic condition, the disease often requires acute hospital care. Patients are commonly admitted for acute pulmonary congestion accompanied by serious or severe shortness of breath. Acute care for congestive heart failure accounts for the use of more hospital days than any other cardiac diagnosis, and consumes in excess of 20 billion dollars in the United States annually.

### SUMMARY

In one aspect, a system for monitoring and treating cardiovascular disease in a medical patient is provided. The system comprises a sensor operable to generate a pressure signal indicative of at least one pressure or at least one pressure parameter within the heart, an interrogation system operable to communicate the pressure signal to a location outside of the medical patient, and a monitoring system operable to determine cardiac data of interest to a physician. In one aspect, processed cardiac data can be displayed to the physician at a location convenient to the physician. It will be appreciated that the physician's desired location can be remote from the patient. Further, in another aspect, the displayed cardiac data can include historical data and/or present/historical treatment protocol(s) for the patient.

In a further aspect, the system and/or method for treating cardiovascular disease can include one or more sensors. In one exemplary aspect, the sensor comprises a passive pressure transducer. In another aspect, the sensor can be in pressure communication with a select portion of the patient's vascular system. In a further aspect, the sensor can be located in a select portion of the pulmonary artery. Optionally, and without limitation, the sensor can be placed in one or more of the following locations: a right atrial appendage, a left atrial appendage, a right or left atrium, a pulmonary vein, a pulmonary capillary wedge position, a right ventricle, a left ventricle, an intrathoracic space, a central vein, or any other desired vascular structure.

Optionally, the system and/or method for treating cardiovascular disease can include one or more sensor signals. In one aspect, the sensor signal can include at least one pressure signal. For example and without limitation, the pressure signal can be a pulmonary artery pressure, a central venous blood pressure, a peripheral arterial blood pressure and/or a left atrial pressure.

Another aspect of this invention is a method that comprises implanting at least one pressure sensor in a desired location within the patient and providing an ex-vivo interrogation system and monitoring system that can be configured to optionally affect at least one of: selectively energizing the at one pressure sensor, receiving a return or output signal from the at one pressure sensor, processing the return signal, and displaying processed data derived from the at least one pressure sensor.

The aspects summarized above and described in greater detail below are useful for the treatment of cardiovascular disease, including congestive heart failure (CHF). CHF is an important example of a medical ailment currently not treated with timely, parameter-driven adjustments of therapy, but one that could potentially benefit greatly from such a strategy. Patients with chronic CHF are typically placed on fixed doses of four or five drugs to manage the disease. The drug regimen commonly includes but is not limited to diuretics, vasodilators such as ACE inhibitors or A2 receptor inhibitors, beta-blockers such as Carvedilol, neurohormonal agents such as spironolactone, and inotropic agents usually in the form of cardiac glycosides such as, for example, Digoxin.

It would be far more cost effective, and much better for the patient's health, if chronic CHF could be managed and controlled by the routine administration of appropriate outpatient oral drug therapy rather than by hospital treatment upon the manifestation of acute symptoms. As with all drugs, these agents are to be taken in doses sufficient to ensure their effectiveness. Problematically, however, over-treatment can lead to bradycardia, hypotension, renal impairment, hyponatremia, hypokalemia, worsening CHF, impaired mental functioning, and other adverse conditions. Adding to the challenge of maintaining proper drug dosage is the fact that the optimal dosage will depend on diet, particularly salt and fluid intake, level of exertion, and other variable factors. Adding further to the problem of managing this condition is the fact that patients frequently miss scheduled doses by forgetting to take pills on time, running out of medications, or deciding to stop medications without consulting their physician.

It is important, therefore, that the patient's condition be monitored regularly and thoroughly, so that optimal or near optimal drug therapy can be maintained. Easily obtained measures of a patient's condition are known, such as weight, peripheral blood pressure, subcutaneous edema, temperature, and subjective measures such as fatigue and shortness of breath. Unfortunately, these measures either do not correlate well enough with specific physiological states to serve as a controlling parameter for therapy, or do correlate but change too late for adjustment of oral medications to be effective. Measures that do change specifically, sensitively, and early in response to changes in the patient's condition are known in the art of heart failure management, but monitoring these measures is problematic in that such monitoring typically involves inserting a catheter into the heart or central blood vessels, therefore requiring frequent visits with a caregiver, and resulting in discomfort, inconvenience, expense, and repeated risks.

Thus, it would be advantageous if methods and systems could be devised by which an outpatient's cardiovascular status in general, and congestive heart failure in particular, could be monitored routinely or continuously, without performing an invasive procedure each time, with attendance by a caregiver only when actually required. In a further aspect, it would be further advantageous if such methods and systems included the ability to communicate diagnostic and treatment information promptly to the patient himself. Such feedback would allow the patient to continue or modify his medications, as prescribed by his physician or licensed caregiver, such that optimal therapeutic doses are achieved, generally without the direct intervention of his physician.

For some classes of drugs (e.g., beta blockers, digoxin, calcium antagonists, amiodorone, etc.), the optimal dose for treating heart failure may be associated with, or exaggerate, episodes of excessively lowered resting heart rate (bradycardia) or an inability to adequately increase heart rate in response the body's demand for augmented blood flow (cardiac output), such as occurs with exercise or stress. The latter condition is known as chronotropic incompetence. Inappropriately low heart rate causes fatigue, poor exercise tolerance, and in the worst cases, deteriorating kidney function, low blood pressure and shock. The risk of these potentially serious complications limits the dose of these beneficial drugs that can be safely prescribed.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1 is a block diagram illustrating an exemplary operating environment for performing the discussed methods.

FIG. 2 illustrates an exemplary interrogation system for communicating with the at least one wireless pressure sensor that is positioned within a body.

FIG. 3 is an exemplary block diagram of an exemplary coupling loop assembly for communication with at least one wireless pressure sensor.

FIG. 4A illustrates an exemplary coupling loop that is un-tuned and FIG. 4B illustrates its equivalent circuit.

FIG. 5A illustrates a loop that is tuned and FIG. 5B illustrates its equivalent circuit.

FIG. 6A illustrates a loop terminated into a receiver with high input impedance and

FIG. 6B illustrates its equivalent circuit.

FIG. 7 is a graph that illustrates the comparison of the frequency response for tuned loops and the frequency response for un-tuned loops with high input impedances at the receiver.

FIG. 8 schematically illustrated two stagger tuned loops.

FIG. 9 illustrates the assembly of two stagger-tuned loops for transmitting the energizing signal to the passive electrical resonant circuit of the assembly and one un-tuned loop for receiving the output signal.

FIG. 10A is a graph illustrating an exemplary energizing signal.

FIGS. 10B, 10C and 10D are graphs illustrating exemplary coupled signals.

FIG. 11 is a schematic block diagram of an exemplary base unit of an interrogation system.

FIG. 12 is a schematic block diagram of another exemplary base unit of an interrogation system.

FIGS. 13A and 13B are graphs illustrating exemplary phase difference signals.

FIG. 14 illustrates frequency dithering.

FIG. 15 illustrates phase dithering.

FIG. 16 is a graph illustrating an exemplary charging response of an LC circuit.

FIG. 17 is a partial schematic block diagram of a portion of an embodiment of an exemplary base unit of an interrogation system.

FIG. 18 is a partial schematic block diagram of a portion of an embodiment of an exemplary base unit of an interrogation system.

FIG. 19 is a block diagram of the system architecture of one exemplary embodiment of the monitoring system.

FIG. 20 is a block diagram of the system architecture of one exemplary embodiment of the home system.

FIG. 21 is an illustration comparing different embodiments of the home system.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the Examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions. It is also contemplated that the computer systems or electronic signal processing systems can be integrated within one or more separate modules.

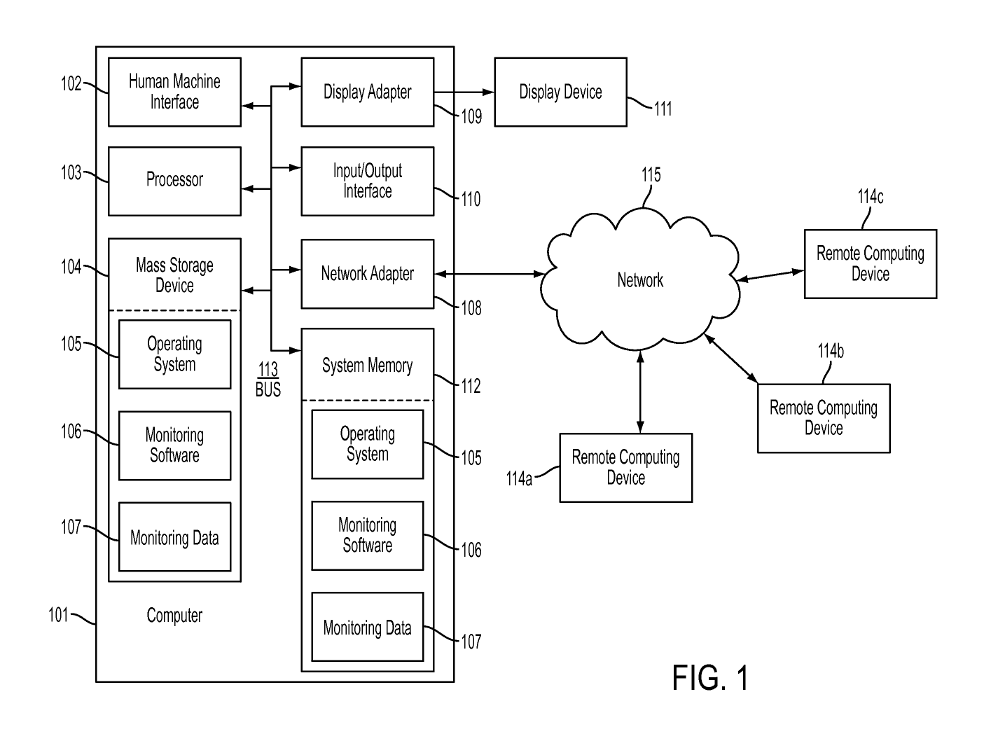

The system has been described above as comprised of units. One skilled in the art will appreciate that this is a functional description and that the respective functions can be performed by software, hardware, or a combination of software and hardware. A unit can be software, hardware, or a combination of software and hardware. The units can comprise the monitoring software 106 as illustrated in FIG. 1 and described below. In one exemplary aspect, the units can comprise a computer 101 as illustrated in FIG. 1 and described below.

FIG. 1 is a block diagram illustrating an exemplary operating environment for performing the disclosed methods. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment. The present methods and systems can be operational with numerous other general purpose or special purpose computing system environments or configurations. Examples of well known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed methods can also be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the systems and methods disclosed herein can be implemented via a general-purpose computing device in the form of a computer 101. The components of the computer 101 can comprise, but are not limited to, one or more processors or processing units 103, a system memory 112, and a system bus 113 that couples various system components including the processor 103 to the system memory 112. In the case of multiple processing units 103, the system can utilize parallel computing.

The system bus 113 represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 113, and all buses specified in this description can also be implemented over a wired or wireless network connection and each of the subsystems, including the processor 103, a mass storage device 104, an operating system 105, monitoring software 106, monitoring data 107, a network adapter 108, system memory 112, an Input/Output Interface 110, a display adapter 109, a display device 111, and a human machine interface 102, can be contained within one or more remote computing devices 114a,b,c at physically separate locations, connected through buses of this form, in effect implementing a fully distributed system. It is of course contemplated that the monitoring system can be distributed over one or more separate components of the system and that the interrogation system can comprise on of the separate components.

The computer 101 typically comprises a variety of computer readable media. Exemplary readable media can be any available media that is accessible by the computer 101 and comprises, for example and not meant to be limiting, both volatile and non-volatile media, removable and non-removable media. The system memory 112 comprises computer readable media in the form of volatile memory, such as random access memory (RAM), and/or non-volatile memory, such as read only memory (ROM). The system memory 112 typically contains data such as monitoring data 107 and/or program modules such as operating system 105 and monitoring software 106 that are immediately accessible to and/or are presently operated on by the processing unit 103.

In another aspect, the computer 101 can also comprise other removable/non-removable, volatile/non-volatile computer storage media. By way of example, FIG. 1 illustrates a mass storage device 104 which can provide non-volatile storage of computer code, computer readable instructions, data structures, program modules, and other data for the computer 101. For example and not meant to be limiting, a mass storage device 104 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 104, including by way of example, an operating system 105 and monitoring software 106. Each of the operating system 105 and monitoring software 106 (or some combination thereof) can comprise elements of the programming and the monitoring software 106. Monitoring data 107 can also be stored on the mass storage device 104. Monitoring data 107 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple systems.

In another aspect, the user can enter commands and information into the computer 101 via an input device (not shown). Examples of such input devices comprise, but are not limited to, a keyboard, pointing device (e.g., a “mouse”), a microphone, a joystick, a scanner, tactile input devices such as gloves, and other body coverings, and the like These and other input devices can be connected to the processing unit 103 via a human machine interface 102 that is coupled to the system bus 113, but can be connected by other interface and bus structures, such as a parallel port, game port, an IEEE 1394 Port (also known as a Firewire port), a serial port, or a universal serial bus (USB).

In yet another aspect, a display device 111 can also be connected to the system bus 113 via an interface, such as a display adapter 109. It is contemplated that the computer 101 can have more than one display adapter 109 and the computer 101 can have more than one display device 111. For example, a display device can be a monitor, an LCD (Liquid Crystal Display), or a projector. In addition to the display device 111, other output peripheral devices can comprise components such as speakers (not shown) and a printer (not shown) which can be connected to the computer 101 via Input/Output Interface 110. Any step and/or result of the methods can be output in any form to an output device. Such output can be any form of visual representation, including, but not limited to, textual, graphical, animation, audio, tactile, and the like.

The computer 101 can operate in a networked environment using logical connections to one or more remote computing devices 114a,b,c. By way of example and not meant to be limiting, a remote computing device can be the interrogation system, a personal computer, portable computer, a server, a router, a network computer, a peer device or other common network node, and so on. Logical connections between the computer 101 and a remote computing device 114a,b,c can be made via a local area network (LAN) and a general wide area network (WAN). Such network connections can be through a network adapter 108. A network adapter 108 can be implemented in both wired and wireless environments. Such networking environments are conventional and commonplace in offices, enterprise-wide computer networks, intranets, and the Internet 115.

For purposes of illustration, application programs and other executable program components such as the operating system 105 are illustrated herein as discrete blocks, although it is recognized that such programs and components reside at various times in different storage components of the computing device 101, and are executed by the data processor(s) of the computer. An implementation of monitoring software 106 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any methods or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The methods and systems can employ Artificial Intelligence techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

In one aspect, a system 10 for monitoring and treating cardiovascular disease in a medical patient is provided. The system comprises a sensor 20 operable to generate a pressure signal indicative of at least one pressure or at least one pressure parameter within the heart, an interrogation system 40 operable to communicate the pressure signal to a location outside of the medical patient, and a monitoring system 60 operable to determine cardiac data of interest to a physician. In one aspect, processed cardiac data can be displayed to the physician at a location convenient to the physician. It will be appreciated that the physician's desired location can be remote from the patient. Further, in another aspect, the displayed cardiac data can include historical data and/or present/historical treatment protocol(s) for the patient.

In a further aspect, the systems and/or method for treating cardiovascular disease can include at least one sensor 20. In one exemplary aspect, the sensor comprises passive pressure transducer. In one aspect, the sensors can be systems that are configured to sense physiological pressure information in unique ways that enable effective management of patient's cardiovascular care and treatment. Desired characteristics desired comprise high fidelity, long-duration, and remote sensing of patients in ambulatory environments.

In one aspect, the sensor 20 is designed to generate a sensor signal that is indicative of a fluid pressure within a desired portion of the patient's vasculature. In one exemplary aspect, fluid pressure within the pulmonary artery is an excellent indicator for quantifying the severity of congestive heart failure, and for assessing the effectivity of drug therapy for treating congestive heart failure.

A particularly effective system and sensor for measurement of hemodynamic parameters is the CardioMEMS™ pressure sensor. As described by U.S. Pat. No. 7,699,059 entitled “Implantable Wireless Sensor,” U.S. Pat. No. 7,679,355 entitled “Communicating with an Implanted Wireless Sensor,” and commonly assigned U.S. patent application Ser. Nos. 12/349,606, 12/175,803, 11/717,967, 11/613,645, 11/472,905, 11/276,571, 11/157,375, 11/105,294, and 10/943,772, which are incorporated in their entirety by reference herein, these pressure sensors are MEMS-based sensors that can be implanted in the pulmonary artery, more particularly in the distal pulmonary artery branch, with a right heart catherization or as part of a graft, such as a AAA stent-graft, and are configured to be energized with RF energy to return high-frequency, high-fidelity dynamic pressure information from a precisely-selected location within a patient's body.

Advantages of the exemplified passive pressure sensor 20 when used in therapeutic development are that it is wireless, which makes it non-invasive after initial implantation, small enough to be implanted in a range of lumens and/or locations within a patient, and it is permanent or can be implanted for prolonged durations. Compare this to RHC's that must typically be inserted in a hospital setting and remain there during measurement.

Another advantage of the exemplified pressure sensor is that it can make measurements during ambulatory activities away from the hospital that are more representative of living conditions of a patient who is going to use a therapeutic. In one aspect, the pressure sensor is non-invasive after implantation, which allows for a patient's ambulatory use, and can be energized via an easy-to-use RF transmitter within a pillow or wand that energizes the sensor.

In a further aspect, the pressure sensor 20 is configured to communicate its monitoring data wirelessly to an interrogator node that is local to the patient. In one aspect, the interrogation system 40 can be configured to process at least a portion of the monitoring data and/or transmit the information over the network to the monitoring system 60 with little or no involvement of the patient, as will be described in more detail below. In addition, this data can be collected in “real time” at “remote” locations. The “real time” collection of data allows physicians to have almost instantaneous access to monitored data via transmission to a range of devices. These devices can comprise, for example and without limitation, a physician's PDA (e.g., BLACKBERRY) or access through a secure website. For “remote” collection of data, the monitoring occurs at the patient's home (or elsewhere) without geographic limitation with respect to the monitoring physician.

In one aspect, the pressure sensor 20 comprises a passive (no battery) LC resonant circuit. Conventionally, a passive LC resonant circuit is composed of two electrical passive components that are connected in series: (a) a coil, or inductor (“L”), (b) a capacitor (“C”). Such a passive electrical circuit exhibits electrical resonance when subjected to an alternating electromagnetic field. The electrical resonance is particularly acute for a specific frequency value or range of the impinging signal. When the impinging signal substantially reaches the resonant frequency of the LC resonant circuit inside the sensor, a pronounced disturbance of the field can be detected wirelessly. In the simplest approximation, the electrical resonance occurs for a frequency f, related to the value of L and C according to Equation 1:

f=(2π(LC)^{1/2})⁻¹ (Equation 1)

The passive electrical resonant circuit for the sensor assemblies described herein that utilize a passive electrical resonant circuit can be fabricated via conventional MEMS approach to sensor design, which lends itself to the fabrication of small sensors that can be formed using, for example and without limitation, biocompatible polymers, ceramics and the like as substrate materials. In a further aspect, appropriately biocompatible coatings can be applied to the surfaces of the respective assemblies in order to prevent adhesion of biological substances to the respective assemblies that could interfere with their proper function.

In one example, the passive electrical resonant circuit of the sensor assembly can be manufactured using micro-machining techniques that were developed for the integrated circuit industry. An example of this type of sensor features an inductive-capacitive (LC) resonant circuit with a variable capacitor is described in Allen et al., U.S. Pat. No. 6,111,520, which is incorporated herein by reference. In this sensor, the capacitance varies with the pressure of the environment in which the capacitor is placed. Consequently, the resonant frequency of the exemplary LC circuit of the Allen pressure sensor varies depending on the pressure of the environment.

As described above, it is contemplated that the LC resonant circuit can comprise a coil inductor operably coupled to a capacitor. In various aspects, the inductance of the LC resonant circuit can be between about 0.1 to about 1000 micro-Henry, preferably between about 1 to about 100 micro-Henry, and more preferably between about 5 to about 15 micro-Henry. The capacitance of the LC resonant circuit can be between about 0.1 to about 1000 pF, preferably between about 0.5 to about 100 pF, and more preferably between about 1 to about 20 pF. The resonant frequency of the LC resonant circuit can be between about 0.1 to about 450 MHz, preferably between about 1 to about 60 MHz, and more preferably between about 25 to about 45 MHz. In addition, the quality factor at self resonance and the frequency range of the self-resonant frequency itself can be between about 5 to 120, preferably between about 5 to about 80, and more preferably between about 10 to about 70.

In one aspect, the coil inductor of the LC resonant circuit can be a substantially planar spiral inductor. Optionally, the coil inductor of the LC resonant circuit can have a longitudinal axis and the respective windings of the coil inductor can spiral about and extend along the longitudinal axis. In this aspect, at least a portion of each winding of the coil is non-planer with respect to the longitudinal axis. For example, in a representative cross-sectional plane that is substantially transverse to the longitudinal axis, portions of the windings in the y-axis can be below the cross-sectional plane and portions of the winding in the y-axis can be above the cross-sectional plane.

In one aspect, the inductor coil can be comprised of the inductor coil body and the coil leads. One skilled in the art will appreciate that numerous parameters of the inductor coil can be varied to optimize the balance of size and the electrical properties of the circuit, including the materials, coil diameter, wire gage, number of coil windings, and cross-sectional area of the coil body. Typically, the material of the coil must be highly conductive and also biocompatible. Suitable materials include, but are not limited to, gold, copper and alloys thereof. If the wire is sufficiently strong, the coil can be self-supporting, also known as an “air core” configuration. A solenoid coil is another suitable configuration. If the wire is not sufficiently strong to be unsupported to maintain its intended configuration during assembly and in use, the coil can be formed around a central bobbin comprised of a suitable dielectric material. In the alternative, the wound coil can be encased in a liquid polymer that can cure or otherwise harden after it is applied to the coil body. Polyimide is one preferred material for this application because of its thermal, electrical, and mechanical properties. However, processes achieving substantially similar results that involve lower processing temperatures would make other polymer choices desirable, such choices being obvious to one skilled in the art.

Optionally, it is contemplated that the passive electrical circuit of the sensor can be housed within a substantially non-permeable enclosure or housing to ensure the protection of the passive electrical circuit of the sensor when the respective sensor is positioned within the living being. In this aspect, the passive electrical circuit of the sensor can be protected from deleterious agents such as corrosion, parasitic excessive strain/stress, biological response, etc. As one will appreciate, it is contemplated that the enclosure can be formed of materials that substantially prevent any undesired fluids and/or gases from passing or diffusing through the walls of the enclosure, utilizing manufacturing processes that eliminate undesired holes that could otherwise permit such passing of undesired fluids or gases.

In another aspect, the enclosure can be formed of materials that do not allow any undesired fluids and/or gases from passing or diffusing through the walls of the enclosure. Exemplary enclosure material can include, without limitation, biocompatible polymer (such as, for example and without limitation, PEAK, PE, PTFE, FEP, semi-crystalline thermoplastic polymers, and the like), glass, fused-silica, low temperature glass, ceramics, quartz, Pyrex, sapphire, sintered zirconia and the like. In one aspect, the housing can be formed from a plurality of glass substrates that are subsequently joined using thermal bonding to form a monolithic sensor housing. Optionally, the level of permeability can be a rate of fluid ingress or egress that changes the original capacitance of the LC circuit by an amount preferably less than 10 percent, more preferably less than 5 percent, and most preferably less than 1 percent over the accumulated time over which measurements will be taken.

Optionally, it is also contemplated that the housing can define an internal cavity in which at least a portion of the passive electrical circuitry can be disposed. In a further aspect, a known and invariant quantity of gas can be added to the internal cavity of the housing. In another aspect, it is contemplated that the enclosure can be formed of materials that will not allow the resonant circuit of the pressure sensor to flex in response to relative motion of the implant that the sensor is mounted thereon or other forces that can be otherwise applied to the sensor. In yet another aspect, the passive circuits comprising a first sensor and additional sensors can be housed in a single enclosure.

Q factor (Q) is the ratio of energy stored versus energy dissipated. The reason Q is important is that the ring down rate of the sensor is directly related to the Q. If the Q is too small, the ring down rate occurs over a substantially shorter time interval. This necessitates faster sampling intervals, making sensor detection more difficult. Also, as the Q of the sensor increases, so does the amount of energy returned to external electronics. Thus, in one aspect, the sensor can be configured with values of Q sufficiently high enough to avoid unnecessary increases in complexity in communicating with the at least one pressure sensor via external electronics. In one aspect, the Q of the sensor can be dependent on multiple factors such as, for example and without limitation, the shape, size, diameter, number of turns, spacing between the turns and cross-sectional area of the inductor component. In addition, Q will be affected by the materials used to construct the pressure sensor. In one example, the sensor can be formed from materials with low loss tangents to affect a sensor with higher Q factors.

In another aspect, the exemplary enclosure materials help to provide the desired biocompatibility, non-permeability and/or manufacturing processing capabilities of the sensor containing the resonant circuit. These exemplary materials are considered dielectrics, that is, they are poor conductors of electricity but are efficient supporters of electrostatic or electroquasistatic fields. A dielectric material has the ability to support such fields while dissipating minimal energy. In this aspect, the lower the dielectric loss, the lower the proportion of energy lost, and the more effective the dielectric material is in maintaining high Q.

With regard to operation within the human body, there is a second issue related to Q, namely that blood and body fluids are conductive mediums and are thus particularly lossy. As a consequence, when a sensor having a resonant circuit is immersed in a conductive fluid, energy from the sensor will dissipate, substantially lowering the Q and reducing the pressure sensor-to-electronics distance. In one aspect, the loss can be minimized by further separation of the sensor having the resonant circuit from the conductive liquid, which can be accomplished, for example and without limitation, by coating at least a portion of the sensor having the resonant circuit in a suitable low-loss-tangent dielectric material.

In another aspect, the system 10 can be configured to provide a monochromatic blast of EM energy and to determine the resonant frequency and bandwidth using an impedance approach. In this approach, an initial frequency that is outside the frequency range of the sensing LC circuit is selected in order to energize the sensor. Then, an excitation signal is transmitted using a transmitting antenna to electromagnetically couple the passive sensing LC circuit to the transmitting antenna, which results in the modification of the impedance of the transmitting antenna. The measured change in impedance of the transmitting antenna allows for the determination of the resonant frequency and bandwidth of the passive sensing LC circuit. In one aspect, as the sensor is activated during the duration of the excitation signal, the impedance of the transmitting antenna, coupled with the known RC time constants of the respective sensors provides a means to determine the resonant frequency and bandwidth of each individual sensor.

In another aspect, the system 10 described herein provides for a system capable of determining the resonant frequency and bandwidth of the sensor 20 using an impedance approach. In this approach, an excitation signal can be transmitted using a transmitting antenna to electromagnetically couple a sensor having a passive electrical resonant circuit to the transmitting antenna, which resultantly modifies the impedance of the transmitting antenna. The measured change in impedance of the transmitting antenna allows for the determination of the resonant frequency and bandwidth of the passive electrical resonant circuit of the sensor.

In a further aspect, the system 10 described herein provides for a transmit and receive interrogation system configured to determine the resonant frequency and bandwidth of a resonant circuit within a particular sensor 20. In this exemplary process, an excitation signal of white noise or predetermined multiple frequencies can be transmitted from a transmitting antenna and the passive electrical resonant circuit of the sensor is electromagnetically coupled to the transmitting antenna. A current is induced in the passive electrical resonant circuit of the sensor as it absorbs energy from the transmitted excitation signal, which results in the oscillation of the passive electrical circuit at its resonant frequency. A receiving antenna, which can also be electromagnetically coupled to the transmitting antenna, receives the excitation signal minus the energy which was absorbed by the sensor. Thus, the power of the received or output signal experiences a dip or notch at the resonant frequency of the sensor. The resonant frequency and bandwidth can be determined from this notch in the power.

In one aspect, the transmit and receive methodology of determining the resonant frequency and bandwidth of a passive electrical resonant circuit of an sensor 20 can include transmitting a frequency signal, such as, without limitation, a multiple frequency signal or a swept frequency signal, from a transmitting antenna to electromagnetically couple the passive electrical resonant circuit on the sensor to the transmitting antenna in order to induce a current in the passive electrical resonant circuit of the sensor. A modified transmitted signal due to the induction of current in the passive electrical circuit is received and processed to determine the resonant frequency and bandwidth.

In another aspect, the system 10 can determine the resonant frequency and bandwidth of a passive electrical resonant circuit within a particular sensor 12 by using a chirp interrogation system, which provides for a transmitting antenna that is electromagnetically coupled to the resonant circuit of the sensor. In this aspect, an excitation signal of white noise or predetermined multiple frequencies can be applied to the transmitting antenna for a predetermined period of time to induce a current in the passive electrical resonant circuit of the sensor at the resonant frequency. The system then listens or otherwise receives an output signal that radiates from the energized passive electrical resonant circuit of the sensor. In this aspect, the resonant frequency and bandwidth of the passive electrical resonant circuit can be determined from the output signal.

In this aspect, the chirp interrogation method can include transmitting a multi-frequency signal pulse from a transmitting antenna; electromagnetically coupling a passive electrical resonant circuit on a sensor to the transmitting antenna to induce a current in the resonant circuit; listening for and receiving an output signal radiated from the energized passive electrical signal of the sensor; determining the resonant frequency and bandwidth from the output signal, and resultantly, determining the measured characteristic acting on the respective sensor from the determined resonant frequency and bandwidth.

In a further aspect, the system 10 described herein can provide an analog system and method for determining the resonant frequency of a passive electrical resonant circuit within a particular sensor 20. The analog system can comprise a transmitting antenna coupled as part of a tank circuit, which, in turn, is coupled to an oscillator. In this aspect, a signal is generated which oscillates at a frequency determined by the electrical characteristics of the tank circuit. The frequency of this signal is further modified by the electromagnetic coupling of the passive electrical resonant circuit of the sensor. This signal can be applied to a frequency discriminator that provides a signal from which the resonant frequency of the resonant circuit can be determined. In this aspect, the analog method can include generating a transmission signal using a tank circuit that includes a transmitting antenna; modifying the frequency of the transmission signal by electromagnetically coupling the passive electrical resonant circuit of the sensor to the transmitting antenna; and converting the modified transmission signal into a standard signal for further application.

One exemplary method of interrogation is explained in more detail in commonly assigned U.S. patent application Ser. No. 11/105,294. In the described methodology, the interrogation system 40 energizes the sensor 20 having the resonant circuit with a low duty cycle, gated burst of RF energy having a predetermined frequency or set of frequencies and a predetermined amplitude. The energizing signal is coupled to the passive electrical resonant circuit via a magnetic loop. The energizing signal induces a current in the passive electrical resonant circuit that is maximized when the frequency of the energizing signal is substantially the same as the resonant frequency of the passive electrical resonant circuit. The system receives the ring down response of the sensor via magnetic coupling and determines the resonant frequency of the sensor, which is then used to determine the measured characteristic acting on the respective sensor. In one aspect, the resonant frequency of the sensor is determined by adjusting the frequency of the energizing signal until the phase of the ring down signal and the phase of a reference signal are equal or at a constant offset. In this manner, the energizing signal frequency is locked to the sensor's resonant frequency and the resonant frequency of the sensor is known. The relative measured characteristic can then be ascertained.

In one aspect, the system can comprise a coupling loop that can be selectively positioned relative to the sensor to maximize the electromagnetic coupling between the passive electrical resonant circuit of the sensor and the coupling loop. The system can also provide the necessary isolation between the energizing signal and the output signal. In one aspect, it is contemplated that the system can energize the passive electrical resonant circuit of the sensor with a low duty cycle, gated burst of RF energy having a predetermined frequency or set of frequencies and a predetermined amplitude. The energizing signal can be electromagnetically coupled to the passive electrical resonant circuit of the sensor via one or more energizing loops. In operation, each energizing loop can be tuned to a different resonant frequency. The selection of the desired resonant frequencies can be based on the desired bandwidth, which, in one aspect of the invention and without limitation can range between about 30 to about 37.5 MHz.

The energizing signal induces a current in the passive electrical resonant circuit of the sensor that is maximized when the energizing frequency is the same as the resonant frequency of the passive electrical resonant circuit of the sensor. The system receives the ring down response of the sensor (or sensors) via one or more coupling loops and determines the resonant frequency of the sensor, which can be used to calculate the measured characteristic acting on the respective sensor.

In one aspect, a pair of phase locked loops (“PLLs”) can be used to adjust the phase and the frequency of the energizing signal until its frequency locks to the resonant frequency of the passive electrical resonant circuit of the sensor. In one embodiment, one PLL samples during the calibration cycle and the other PLL samples during the measurement cycle. In one non-limiting example, these cycles can alternate every 10 microseconds and can be synchronized with the pulse repetition period. In one aspect, the calibration cycle adjusts the phase of the energizing signal to a fixed reference phase to compensate for any system delay or varying environmental conditions. The environmental conditions that can affect the accuracy of the reading can include, but are not limited to, proximity of reflecting or magnetically absorbative objects, variation of reflecting objects located within transmission distance, variation of temperature or humidity which can change parameters of internal components, and aging of internal components.

In one aspect, one of the PLLs can be used to adjust the phase of the energizing signal and is referred to herein as the fast PLL. The other PLL can be used to adjust the frequency of the energizing signal and is referred to herein as the slow PLL. During the time that the energizing signal is active, a portion of the signal enters the receiver and is referred to herein as a calibration signal. The calibration signal is processed and sampled to determine the phase difference between its phase and the phase of a local oscillator. The cycle in which the calibration signal is sampled is referred to as the calibration cycle. In one aspect, the system can adjust the phase of the energizing signal to drive the phase difference to zero or another select reference phase.

During the measurement cycle, the signal coupled from the passive electrical resonant circuit of the sensor (referred to herein as the output signal) can be processed and sampled to determine the phase difference between the output signal and the energizing signal. The system can then adjust the frequency of the energizing signal to drive the phase difference to zero or other reference phase. Once the slow PLL is locked, the frequency of the energizing signal is deemed to match the resonant frequency of the passive electrical resonant circuit of the sensor. The operation of the slow PLL is qualified based on signal strength so that the slow PLL does not lock unless the strength of the output signal meets a predetermined signal strength threshold.

In one aspect, a single un-tuned coupling loop can be is used. In this exemplary aspect, the loop can be connected to an input impedance that is high relative to the loop inductance. Optionally, multiple coupling loops can be used and each loop is tuned to a different resonant frequency.

In another aspect, the loops can be connected to a base unit 102 that generates the energizing signal and processes the output signal via a cable assembly. In this aspect, the cable assembly provides isolation between the energizing signal and the sensor signal by maximizing the distance between the coaxial cables that carry the signals and maintaining the relative positions of the coaxial cables throughout the cable assembly. In another exemplary aspect, the coaxial cables can be positioned on opposite sides of an internal cable, approximately 180 degrees apart. Shielding can also be used to isolate the energizing signal from the output signal. In one aspect, it is contemplated that additional shielding can be provided around each of the respective coaxial cables.

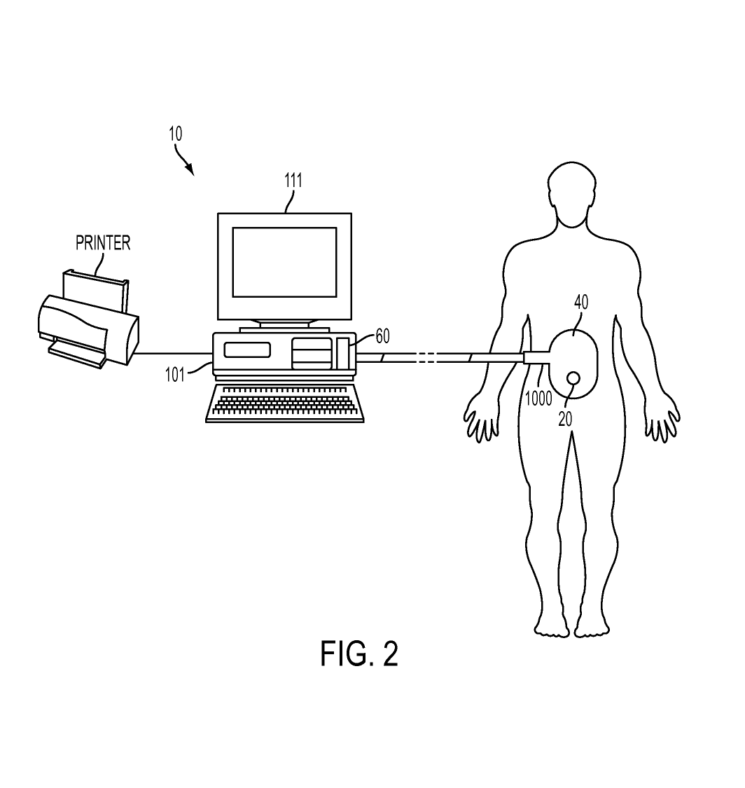

In one aspect, FIG. 2 illustrates an exemplary interrogation system for communicating with the wireless apparatus described above that is positioned within a body. Without limitation, it is contemplated that the system can be used in at least two environments: the operating room during implant and the physician's office during follow-up examinations.

In one exemplary embodiment, the interrogation system 40 can comprise at least one of a coupling loop 1000, the base unit 1002, a display device 1004, and an input device 1006, such as, for example and without limitation, a keyboard. In one exemplary embodiment, the base unit can include an RF amplifier, a receiver, and signal processing circuitry. In one aspect, the coupling loop 1000 can be configured to charge the passive electrical resonant circuit of the sensor and then couple signals from the energized passive electrical resonant circuit of the sensor into the receiver. Schematic details of the exemplary circuitry are illustrated in FIG. 2.

The display 1004 and the input device 1006 can be used in connection with the user interface for the system. In the embodiment illustrated in FIG. 2, the display device and the input device are conventionally connected to the base unit. In this embodiment, the base unit can also provides conventional computing functions. In other embodiments, the base unit can be connected to the monitoring system or a conventional computer, such as a laptop, via a communications link, such as an RS-232 link. If a separate computer is used, then the display device and the input devices associated with the computer can be used to provide the user interface.

In one aspect, LABVIEW software can be used to provide the user interface, as well as to provide graphics, store and organize data and perform calculations for calibration and normalization. The user interface can record and display patient data and guide a user through surgical and follow-up procedures. In another aspect, an optional printer 1008 can be operably connected to the base unit and can be used to print out patient data or other types of information. As will be apparent to those skilled in the art in light of this disclosure, other configurations of the system, as well as additional or fewer components can be utilized with embodiments of the invention.

In one embodiment, the coupling loop can be formed from a band of copper. In this aspect, it is contemplated that the coupling loop comprises switching and filtering circuitry that is enclosed within a shielded box. The loop can be configured to charge the passive electrical resonant circuit of the sensor and then couple signals from the energized passive electrical resonant circuit of the sensor into a receiver. It is contemplated that the antenna can be shielded to attenuate in-band noise and electromagnetic emissions.

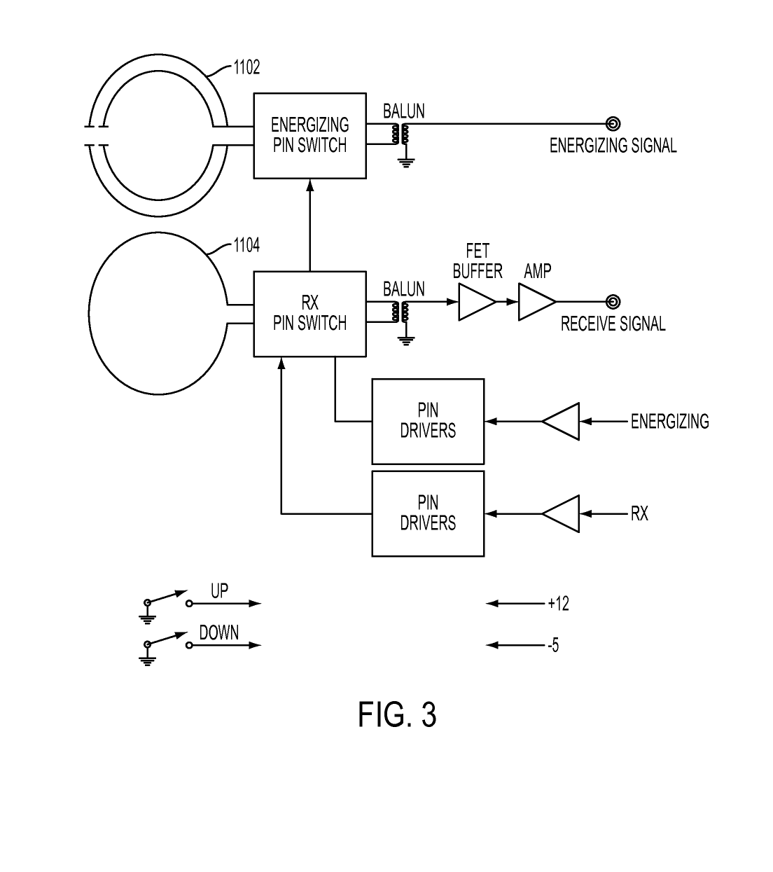

In an alternative embodiment for a coupling loop, as shown in FIG. 3, separate loops for energizing 1102 and for receiving 1104 are provided, although a single loop can be used for both functions. PIN diode switching inside the loop sensor can be used to provide isolation between the energizing phase and the receive phase by opening the RX path pin diodes during the energizing period, and opening the energizing path pin diodes during the coupling period. It is contemplated in this embodiment that multiple energizing loops can be staggered tuned to achieve a wider bandwidth of matching between the transmit coils and the transmit circuitry.

In one aspect, the coupling loop or antenna can provide isolation between the energizing signal and the output signal, support sampling/reception of the output signal soon after the end of the energizing signal, and minimize switching transients that can result from switching between the energizing and the coupled mode. The coupling loop can also provide a relatively wide bandwidth, for example and without limitation, from between about 30 to about 37.5 MHz.

In one embodiment, separate loops can be used for transmitting the energizing signal to the passive electrical resonant circuit of the sensor and coupling the output signal from the energized passive electrical resonant circuit of the sensor. Two stagger-tuned loops can be used to transmit the energizing signal and an un-tuned loop with a high input impedance at the receiver can be used to receive the output signal. The term “coupling loop” is used herein to refer to both the loop(s) used to receive the output signal from the energized passive electrical resonant circuit of the sensor (the “sensor coupling loop”), as well as the loop sensor that includes the loop(s) used to transmit the energizing signal to the passive electrical resonant circuit of the sensor (the “energizing loop”) and the sensor coupling loop(s).

During the measurement cycle, the sensor coupling loop can be configured to couple the output signal from the energized passive electrical resonant circuit of the sensor, which is relatively weak and dissipates quickly. In one aspect, the voltage provided to the receiver in the base unit depends upon the design of the sensor coupling loop and in particular, the resonant frequency of the loop.

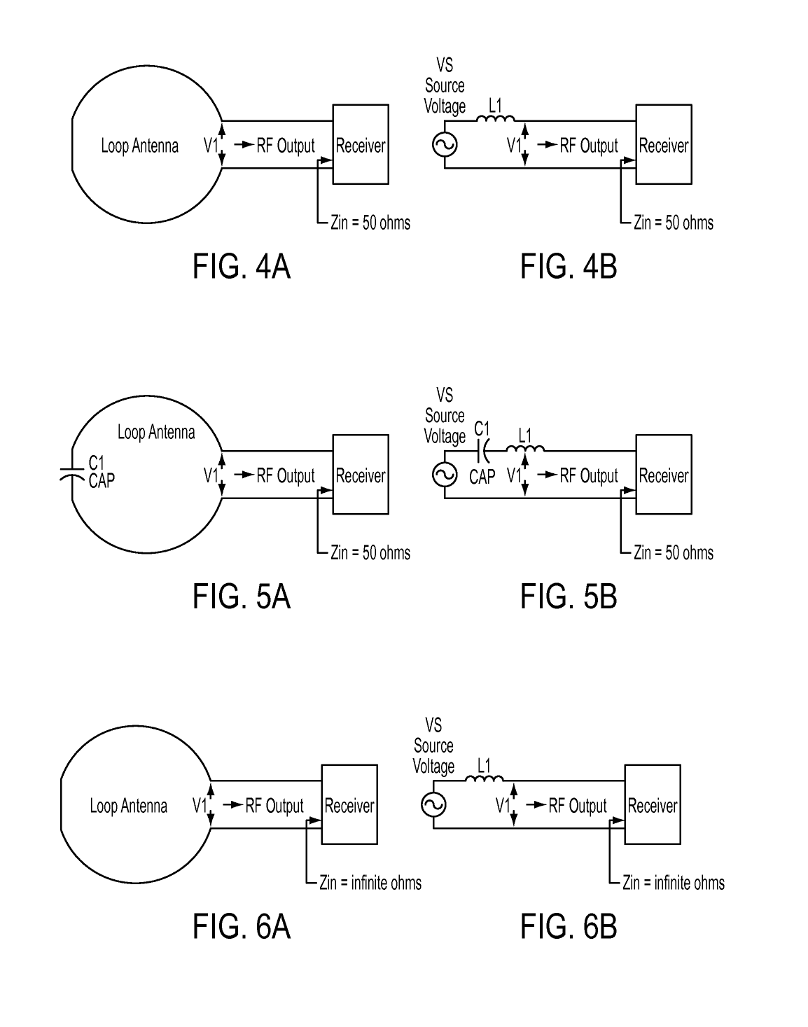

In a further aspect, it is contemplated that the coupling loop can be un-tuned or tuned. FIG. 4A illustrates a loop that is un-tuned and FIG. 4B illustrates its equivalent circuit. The loop has an inductance, L₁, and is terminated into the receiver using a common input impedance, which can, for example and without limitation, be 50 ohms. The voltage at the receiver, V₁, is less than the open circuit voltage of the loop, i.e., the voltage that would be coupled by the loop if the loop was not terminated, Vₛ, and can be calculated as shown below:

$${V}_{1} = {V}_{s} \frac{50}{50 + jω {L}_{1}} \qquad Equation 2$$

Where L1 is the inductance of the loop and ω=2πf, with f=frequency in hertz.

To maximize the voltage at the receiver, it is contemplated that the loop can be tuned. FIG. 5A illustrates a loop that is tuned and FIG. 5B illustrates its equivalent circuit. In this aspect, the loop has an inductance, L₁, and a capacitance, C₁. The capacitance, C₁, is selected so that it cancels the inductance, L₁ at the resonant frequency, i.e., the series resonant circuit, C₁-L₁, is 0 ohms at the resonant frequency. At the resonant frequency the voltage at the receiver, V₁, equals the voltage coupled by the loop, Vₛ. One disadvantage of this type of loop is that it is optimized for a single frequency. If the loop is used in an environment where the frequency of the output signal is changing, then the capacitance is either changed dynamically or set to a compromise value (e.g., the loop is tuned to a single frequency within the band of interest).

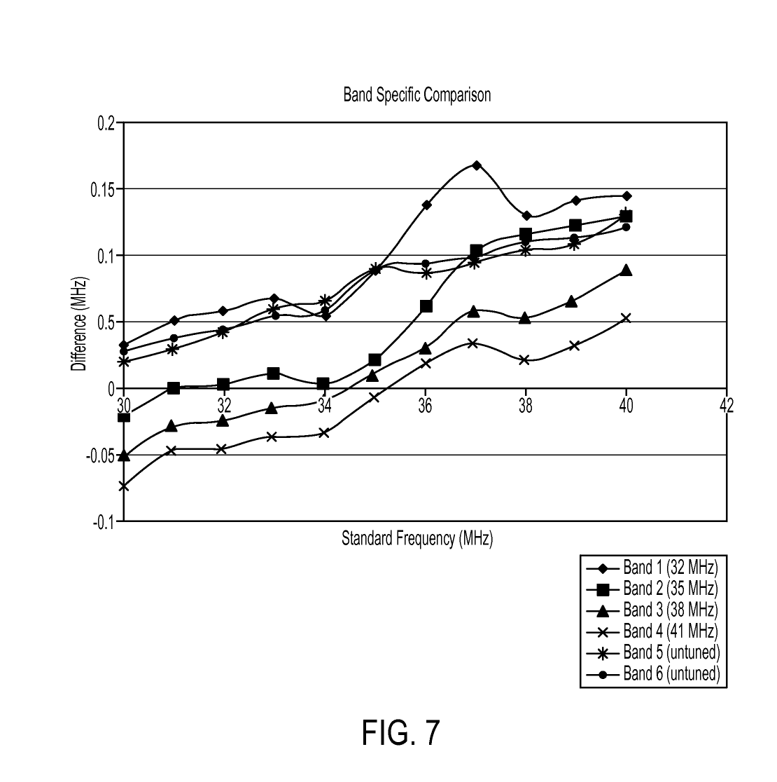

To minimize this issue, another embodiment illustrated in FIGS. 7A and 7B uses an un-tuned loop with a high input impedance at the receiver. FIG. 6A illustrates a loop terminated into a receiver with a high input impedance and FIG. 6B illustrates its equivalent circuit. In this aspect, the input impedance at the receiver is selected so that the energy lost due to the loop impedance, L₁, is relatively insignificant. Using Zin as the input impedance at the receiver, the voltage at the receiver, V₁, is calculated as shown below.

$${V}_{1} = {V}_{s} \frac{Zin}{Zin + jω {L}_{1}} \qquad Equation 3$$

Since Zin is much larger than jωL₁, this can be approximated by the following equation

$${V}_{1} = {V}_{s} \frac{∞}{∞ + jω {L}_{1}} , \\ or \\ {V}_{1} = {V}_{s} \qquad Equation 4$$

As shown by the foregoing equation, the use of a relatively high input impedance at the input of the receiver negates L₁ for all frequencies. In one embodiment, a high impedance buffer can be inserted between the loop and an exemplary 50 ohm receiver circuit. In this embodiment, the high impedance buffer is on the order of 1 Mohm while the impedance of the loop is on the order of 200 ohms. In other embodiments, it is contemplated that the input impedance is at least two times the loop impedance.

In one aspect, the frequency response within the band of interest is more monotonic if the sensor coupling loop uses a high input impedance at the receiver, than if a tuned loop is used with a 50 ohm input impedance. FIG. 7 compares the frequency response for tuned loops and the frequency response for un-tuned loops with high input impedances at the receiver. The y-axis represents the difference in measured frequency between a calibration system using a network analyzer and the loop. The x-axis represents the frequency of the L-C standard used in the measurements. Linear interpolation can be used between measurement points. Band 1 corresponds to a loop resonant at 32 MHz, Band 2 corresponds to a loop resonant at 35 MHz, Band 3 corresponds to a loop resonant at 38 MHz, and Band 4 corresponds to a loop resonant at 41 MHz. Bands 1-4 correspond to a prior art design that uses switched capacitors banks to vary the loop resonance to achieve the needed bandwidth. Bands 5 and 6 correspond to un-tuned loops.

Bands 1-4 illustrate a slope variation within the band of interest, which can affect the accuracy of measurements made using the loop. Bands 5 and 6 illustrate that the variation within the band of interest is less than in the systems using a tuned loop. The more monotonic frequency response of an un-tuned loop with a high input impedance generally requires a simpler set of calibration coefficients to be used for the frequency conversion calculation.

An alternative embodiment to using an un-tuned loop and a high input impedance is to use stagger-tuned loops. If stagger tuned loops are used to receive the output signal, then the loops can be tuned in a manner similar to that described in the following paragraphs in connection with the transmission of an energizing signal.

During the energizing mode, the energizing loop produces a magnetic field. The intensity of the magnetic field produced by the energizing loop depends, in part, on the magnitude of the current within the loop. In one aspect, the current is maximized at the energizing frequency if the impedance of the loop is essentially 0 ohms at the energizing frequency. The resonant frequency of the loop is related to the loop inductance and capacitance, as shown below.

$${f}_{o} = \frac{1}{2 π \sqrt{L * C 1}} \qquad Equation 5$$

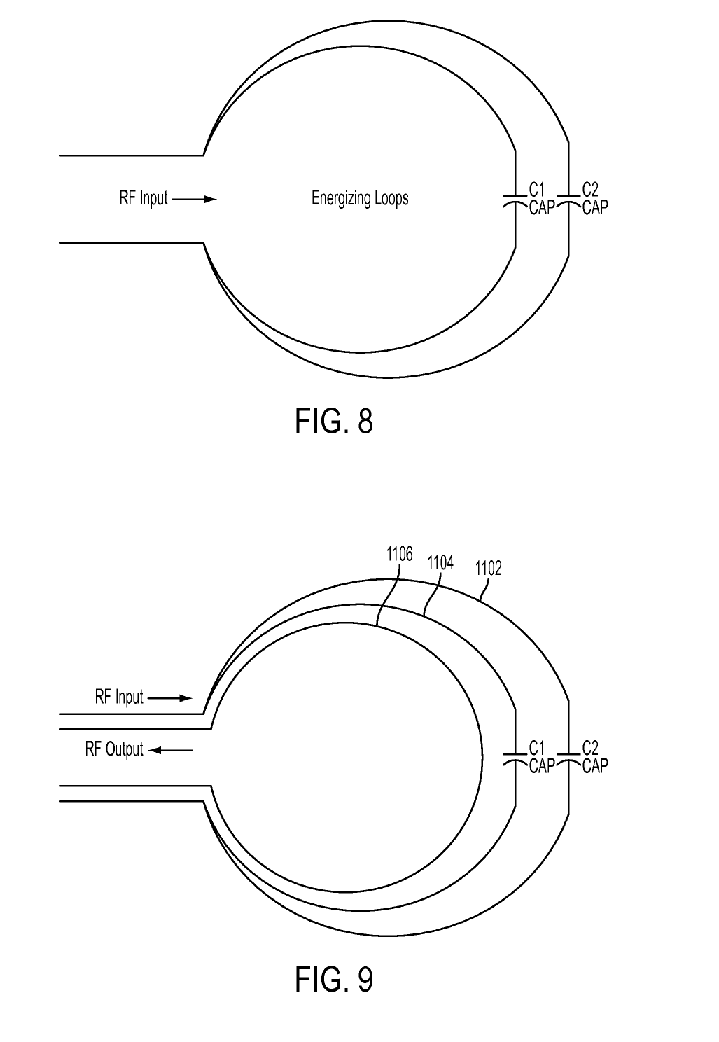

The impedance of the loop is preferably 0 ohms over the frequency range of interest, which, in an exemplary operating environment, can be, without limitation between about 30 MHz to about 37.5 MHz. To achieve the desired impedance over the desired frequency range, two or more loops can be stagger tuned as exemplarily shown in FIG. 8.

The resonant frequencies for the loops are based on the bandwidth of interest. If there are two loops, then the loops can be spaced geometrically. In one exemplary non-limiting aspect, the resonant frequency of the first loop is can be about 31 MHz and the resonant frequency of the second loop can be about 36.3 MHz, which corresponds to the pole locations of a second order Butterworth bandpass filter having about −3 dB points at about 30 MHz and about 37.5 MHz. Although FIG. 8 illustrates two loops, it is contemplated that other embodiments can use a different number of loops, which provides coverage for a much wider frequency range. In one aspect, the loops can be spaced logarithmically if there are more than two loops.

FIG. 9 illustrates the assembly of two stagger-tuned loops 1102, 1104 for transmitting the energizing signal to the passive electrical resonant circuit of the sensor and one un-tuned loop 1106 for receiving the output signal. In this aspect, the loops are parallel to one another with the un-tuned loop inside the stagger-tuned loops. Placing the loop used to receive the output signal inside of the loops used to transmit the energizing signal helps to shield the output signal from environmental interferences. In one embodiment, the loops can be positioned within a housing.

One will appreciate that the signal from an implanted passive sensor is relatively weak and is attenuated by the surrounding tissue and the distance between the sensor and the coupling loop. Optimizing the position and angle of the coupling loop relative to the sensor can help maximize the coupling between the sensor and the coupling loop. In one aspect, the coupling loop can be positioned so that a plane defined by the sensor coupling loop is approximately parallel to the inductor within the passive electrical resonant circuit of the sensor and the sensor is approximately centered within the sensor coupling loop.

In one aspect, isolation of the energizing signal and the output signal provided by the base unit and the coupling loop can be maintained in the cable that connects the base unit to the coupling loop. In one aspect, a cable can connect the base unit to the coupling loop and isolate the energizing signal from the output signal. In one aspect, the distal end of the cable that connects to the base unit can comprise a multi-pin connector (e.g., AL06F15-ACS provided by Amphenol) and a right angle housing. The proximal end of the cable that connects to the coupling loop can comprise a first connector, which can be a multi-pin connector (e.g., AMP 1-87631-0 provided by Amphenol) that operably connects to the filtering and switching circuitry associated with the loop; a second connector that operably connects to the energizing loop; and a third connector that operably connects to the loop that couples the signal from the sensor. In this exemplary aspect, the right angle housing and the strain relief provide strain relief at the respective ends of the cable. When assembled with the housing, the strain relief can be positioned proximate to the housing. Optionally, other types of strain relief can be implemented, including, without limitation, physical constraints, such as tie wraps, ferrals or epoxy, and/or service loops. In one aspect, the cable can also comprise ferrite beads, which can help reduce ground currents within the cable.

In one aspect, the position of the coaxial cables within the cable is designed to maximize the isolation between the energizing signal and the sensor signal, while minimizing the diameter of the cable. The cable is configured to maximize the isolation between the coax cable that transmits the energizing signal and the inner bundle and the twisted pairs and the coax cable that receives the sensor signal and the inner bundle.

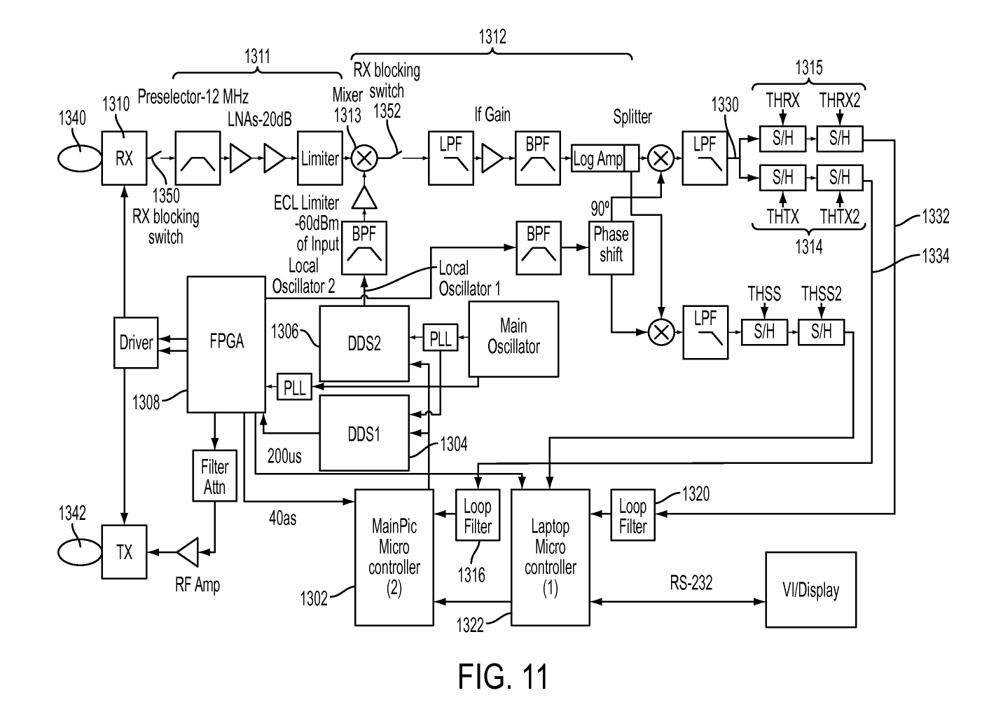

In an alternative embodiment and referring now to FIGS. 11(a)-17, the interrogation system can be configured to determine the resonant frequency of the sensor (and therefore the desired measured characteristic) by adjusting the phase and frequency of an energizing signal until the frequency of this signal locks to the resonant frequency of the sensor. In one aspect, the interrogation system energizes the sensor with a low duty cycle, gated burst of RF energy of a predetermined frequency or set of frequencies and predetermined amplitude. This signal induces a current in the sensor that can be used to track the resonant frequency of the sensor. The interrogation system receives the ring down response of the sensor and determines the resonant frequency of the sensor, which is used to calculate the measured characteristic, such as, for example, pressure, acting thereon the sensor. As described above, interrogation the system can use a pair of PLL's to adjust the phase and the frequency of the energizing signal to track the resonant frequency of the sensor. In one exemplary aspect, the first measurement can be taken during introduction of the sensor for calibration and the second measurement can be taken after placement for functional verification of the sensor.

The interrogation system communicates with the implanted sensor to determine the resonant frequency of the sensor, which can comprise an LC resonant circuit having a variable capacitor. In one exemplary aspect, and not meant to be limiting, the distance between the plates of the variable capacitor varies as the surrounding pressure varies. Thus, the resonant frequency of the circuit can be used to determine the pressure acting thereon the sensor.

In one aspect, the interrogation system can energize the sensor with an RF burst. The energizing signal can be a low duty cycle, gated burst of RF energy of a predetermined frequency or set of frequencies and predetermined amplitude. In one non-limiting example, the duty cycle of the energizing signal can range between about 0.1% to 50%. In another non-limiting example, the interrogation system can energize the sensor with a 30-37.5 MHz fundamental signal at a pulse repetition rate of 100 kHz with a duty cycle of 20%. The energizing signal is coupled to the sensor via a magnetic loop. This signal induces a current in the sensor which has maximum amplitude at the resonant frequency of the sensor. During this time, the sensor charges exponentially to a steady-state amplitude that is proportional to the coupling efficiency distance between the sensor and loop, and the RF power.

FIG. 16 shows the charging response of a typical LC circuit to a burst of RF energy at its resonant frequency. The speed at which the sensor charges is directly related to the Q (quality factor) of the sensor. Therefore, it is contemplated that the “on time” of the pulse repetition duty cycle can be optimized for the Q of the sensor. The system receives the ring down response of the sensor via magnetic coupling and determines the resonant frequency of the sensor.

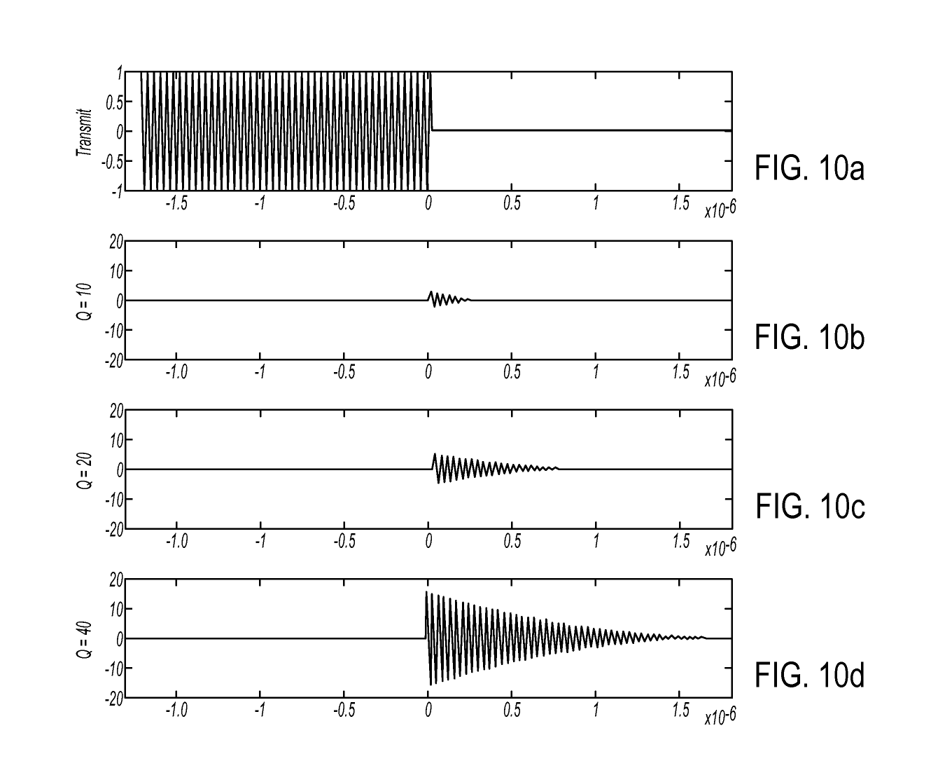

FIG. 10A illustrates a typical energizing signal and FIGS. 10B, 10C and 10D illustrate typical coupled signals for various values of Q (quality factor) for the sensor. When the main unit is coupling energy at or near the resonant frequency of the sensor, the amplitude of the sensor return is maximized, and the phase of the sensor return will be close to zero degrees with respect to the energizing phase. The sensor return signal is processed via phase-locked-loops to steer the frequency and phase of the next energizing pulse.

In a further aspect, FIG. 11 illustrates a schematic diagram of the signal processing components within an exemplary base unit 1002. In one aspect, the base unit determines the resonant frequency of the sensor by adjusting the energizing signal so that the frequency of the energizing signal matches the resonant frequency of the sensor. In the exemplary embodiment illustrated by FIG. 11, two separate processors 1302, 1322 and two separate coupling loops 1340, 1342 are shown. In one embodiment, processor 1302 is associated with the base unit and processor 1322 is associated with a computer connected to the base unit. In other embodiments, it is contemplated that a single processor can be used to provide the same functions as the two separate processors. In other embodiments, it is also contemplated that a single loop can be used for both energizing and for coupling the sensor energy back to the receiver. As will be apparent to those skilled in the art, other configurations of the base unit are possible that use different components.

In one aspect, a pair of PLLs can be used. Is this aspect, the fast PPL is used to adjust the phase of the energizing signal and the slow PLL is used to adjust the frequency of the energizing signal. The base unit 1002 can be configured to provide two cycles: the calibration cycle and the measurement cycle. In one aspect, the first cycle is a 10 microsecond energizing period for calibration of the system, which is referred to herein as the calibration cycle, and the second cycle is a 10 microsecond energizing/coupling period for energizing the sensor and coupling a return signal from the sensor, which is referred to herein as the measurement cycle.

During the calibration cycle, the interrogation system generates a calibration signal for system and environmental phase calibration and during the measurement cycle the system both sends and listens for a return signal, i.e. the sensor ring down. Alternatively, as those skilled in the art will appreciate, it is contemplated that the calibration cycle and the measurement cycle can be implemented in the same pulse repetition period.

The phase of the energizing signal is adjusted during the calibration cycle by the fast PLL and the frequency of the energizing signal is adjusted during the measurement cycle by the slow PLL. The following description of the operation of the PLLs is presented sequentially for simplicity. However, as those skilled in the art will appreciate, the PLLs can operate simultaneously.

Initially the frequency of the energizing signal is set to a default value determined by the calibration parameters of the sensor. Each sensor is associated with a number of calibration parameters, such as frequency, offset, and slope. An operator of the interrogation system enters the sensor calibration parameters into the interrogation system via the user interface and the interrogation system determines an initial frequency for the energizing signal based on the particular sensor. Alternatively, the sensor calibration information could be stored on portable storage devices, bar codes, or incorporated within a signal returned from the sensor. In one aspect, the initial phase of the energizing signal can be arbitrary.

The initial frequency and the initial phase are communicated from the processor 1302 to the DDSs (direct digital synthesizers) 1304, 1306. The output of DDS1 1304 is set to the initial frequency and initial phase and the output of DDS2 1306 (also referred to as local oscillator 1) is set to the initial frequency plus the frequency of the local oscillator 2. In one aspect, the phase of DDS2 is a fixed constant. In one embodiment, the frequency of local oscillator 2 is 4.725 MHz. The output of DDS1 is gated by the field programmable gate array (FPGA) 1308 to create a pulsed transmit signal having a pulse repetition frequency (“PRF”). The FPGA provides precise gating so that the base unit can sample the receive signal during specific intervals relative to the beginning or end of the calibration cycle.

During the calibration cycle, the calibration signal which enters the receiver 1310 is processed through the receive section 1311 and the IF section 1312, and is sampled. In one embodiment, the calibration signal is the portion of the energizing signal that leaks into the receiver (referred to herein as the energizing leakage signal). The signal is sampled during the on time of the energizing signal by a sample and hold circuit 1314 to determine the phase difference between the signal and local oscillator 2. FIG. 11 illustrates two cascaded sample and holds in circuit 1314 to provide both fast sampling and a long hold time. Alternatively, a single sample and hold can be used in circuit 1314. In the embodiment where the calibration signal is the portion of the energizing signal that leaks into the receiver, the signal is sampled approximately 100 ns after the beginning of the energizing signal pulse. Since the energizing signal is several orders of magnitude greater than the coupled signal, it is assumed that the phase information associated with the leaked signal is due to the energizing signal and the phase delay is due to the circuit elements in the coupling loop, circuit elements in the receiver, and environmental conditions, such as proximity of reflecting objects.

The phase difference is sent to a loop filter 1316. The loop filter is set for the dynamic response of the fast PLL. In one embodiment, the PLL bandwidth is 1000 Hz and the damping ratio is 0.7. A DC offset is added to allow for positive and negative changes. The processor 1302 reads its analog to digital converter (A/D) port to receive the phase difference information and adjusts the phase sent to direct digital synthesizer 1 (DDS 1) to drive the phase difference to zero. This process is repeated alternatively until the phase difference is zero or another reference phase.

The phase adjustment made during the energizing period acts to zero the phase of the energizing signal with respect to local oscillator 2. Changes in the environment of the antenna or the receive chain impedance, as well as the phase delay within the circuitry prior to sampling affect the phase difference reading and are accommodated by the phase adjustment.

During the measurement cycle, the energizing signal may be blocked from the receiver during the on time of the energizing signal. During the off time of the energizing signal, the receiver is unblocked and the coupled signal from the sensor is received. The coupled signal is amplified and filtered through the receive section 1311. The signal is down converted and additional amplification and filtering takes place in the IF section 1312. In one embodiment, the signal is down converted to 4.725 MHz. After being processed through the IF section, the signal is mixed with local oscillator 2 and sampled by sample and hold circuits 1315 to determine the phase difference between the coupled signal and the energizing signal. FIG. 11 illustrates two cascaded sample and holds in circuit 1315 to provide both fast sampling and a long hold time. Alternatively, a single sample and hold can be used in circuit 1315. In one embodiment, the sampling can occur approximately 30 ns after the energizing signal is turned off.

In other aspects, group delay or signal amplitude can be used to determine the resonant frequency of the sensor. The phase curve of a second order system passes through zero at the resonant frequency. Since the group delay (i.e., the derivative of the phase curve) reaches a maximum at the resonant frequency, the group delay can be used to determine the resonant frequency. Alternatively, the amplitude of the sensor signal can be used to determine the resonant frequency. The sensor acts like a bandpass filter so that the sensor signal reaches a maximum at the resonant frequency.

The sampled signal is accumulated within a loop filter 1320. The loop filter is set for the dynamic response of the slow PLL to aid in the acquisition of a lock by the slow PLL. The PLLs are implemented with op-amp low pass filters that feed A/D inputs on microcontrollers, 1302 and 1322, which in turn talk to the DDSs, 1304 and 1306, which are in communication with the energizing signal and local oscillator. The microcontroller that controls the energizing DDS 1304 also handles communication with the display. The response of the slow PLL depends upon whether the loop is locked or not. If the loop is unlocked, then the bandwidth is increased so that the loop will lock quickly. In one embodiment, the slow PLL has a damping ratio of 0.7 and a bandwidth of 120 Hz when locked (the Nyquist frequency of the blood pressure waveform), which is approximately ten times slower than the fast PLL.

A DC offset is also added to the signal to allow both a positive and a negative swing. The output of the loop filter is input to an A/D input of processor 1322. The processor determines a new frequency and sends the new frequency to the DSSs. The processor offsets the current frequency value of the energizing signal by an amount that is proportional to the amount needed to drive the output of the slow PLL loop filter to a preset value. In one embodiment the preset value is 2.5V and zero in phase. The proportional amount is determined by the PLL's overall transfer function.

The frequency of the energizing signal is deemed to match the resonant frequency of the sensor when the slow PLL is locked. Once the resonant frequency is determined, the measured characteristic can be calculated using the calibration parameters associated with the respective sensor, which results in a difference frequency that is proportional to the measured characteristic.

The operation of the slow PLL is qualified based on signal strength. The base unit includes signal strength detection circuitry. If the received signal does not meet a predetermined signal strength threshold, then the slow PLL is not allowed to lock and the bandwidth and search window for the PLL are expanded. Once the received signal meets the predetermined signal strength threshold, then the bandwidth and search window of the slow PLL is narrowed and the PLL can lock.

In one aspect, phase detection and signal strength determination can be provided via the “I” (in phase) and “Q” (quadrature) channels of a quadrature mixer circuit. The “I” channel is lowpass filtered and sampled to provide signal strength information to the processing circuitry. The “Q” channel is lowpass filtered and sampled (THSS, THSS2) to provide phase error information to the slow PLL.

The base unit can comprise two switches, RX blocking switches 1350 and 1352, which aid in the detection of the sensor signal. One of the RX blocking switches precedes the preselector in the receive section 1311 and the other RX blocking switch follows the mixer in the IF section 1312. The FPGA controls the timing of the RX blocking switches (control signals not shown). The RX blocking switches are closed during the on time of the energizing signal during the calibration cycle and generally closed during the off time of the energizing signal during the measurement cycle. During the measurement cycle the timing of the RX blocking switches is similar to the timing of the switch that controls the energizing signal into the receiver during the measurement cycle, but the RX blocking switches are closed slightly later to account for signal travel delays in the system. The RX blocking switches prevent the energizing signal that leaks into the receiver during the measurement cycle (specifically during the on time of the energizing signal) from entering the IF section. If the leakage signal enters the IF section, then it charges the IF section and the IF section may not settle out before the sensor signal arrives. For example, in one instance the IF section was charged for several hundred nanoseconds after the on time of the energizing signal. Blocking the leakage signal from the IF section eliminates this problem and improves detection of the sensor signal.

In another embodiment, the base unit can be configured to use multiple sampling points rather than the single sampling point discussed above in connection with FIG. 11. If a single sampling point is used and the sampling point coincides with a point where the average DC voltage of the phase detector is zero, then the system can lock even though the frequency is not the correct frequency. This situation can occur when there is system stress, such as a DC offset in the loop integrator or some other disturbance. The use of multiple sampling points helps prevent a false lock under these circumstances.

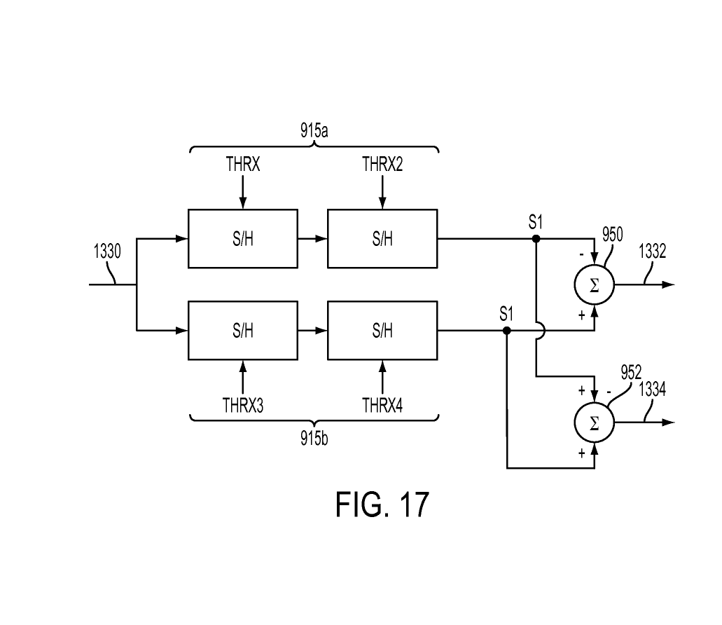

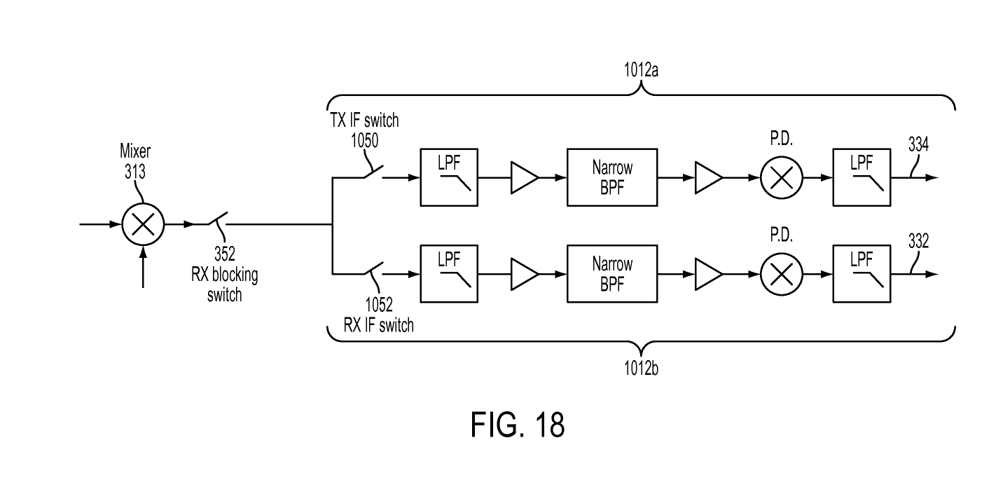

FIG. 17 illustrates a portion of the base unit for an embodiment that uses two sampling points, S1, S2. In this aspect, the components illustrated in FIG. 17 are used instead of the sample and hold components 1314, 1315 used in FIG. 11. As discussed above in connection with FIG. 11, this embodiment uses a pair of PLLs. The phase of the energizing signal is adjusted by the fast PLL and the frequency of the energizing signal is adjusted by the slow PLL. However, in this embodiment only a single cycle is needed to adjust the phase and frequency of the energizing signal, i.e. separate calibration and measurement cycles are not necessary. Since only a single cycle is used, the timing of the RX blocking switches is slightly different than that described above in connection with FIG. 11. In this embodiment, the RX blocking switches are generally closed during the off time of the energizing signal. The specific timing of the closure of the RX blocking switches may be system specific and can be adjusted to account for signal travel delays in the system.

The initial frequency and phase of the energizing signal are set as described above in connection with FIG. 11. The energizing signal may be blocked from the receiver during the on time of the energizing signal. During the off time of the energizing signal, the receiver is unblocked and the coupled signal from the sensor is received. The coupled signal is amplified and filtered through the receive section 1311. The signal is down converted and additional amplification and filtering takes place in the IF section 1312. In one aspect, the signal is down converted to 4.725 MHz. After being processed through the IF section, the signal is mixed with local oscillator 2 and sampled by the two sample and hold circuits 915a and 915b to determine the phase difference between the coupled signal and the energizing signal.

The two sample points are applied to a first differential amplifier 950 and a second differential amplifier 952. The first differential amplifier outputs a signal representing the difference between the two sampling points (S2-S1), which is fed into the loop filter 1320 and used to adjust the frequency of the energizing signal. The second differential amplifier 952 outputs a signal representing the sum of the two sampling points (S1+S2), which is fed into the loop filter 1316 and used to adjust the phase of the energizing signal.

In this aspect, the FPGA controls the timing of the two sample and hold circuits. In one aspect, the first sample point occurs approximately 30 ns after the energizing signal is turned off and the second sample point occurs approximately 100 to 150 ns after the energizing signal is turned off. The timing of the first sampling point can be selected so that the first sampling point occurs soon after the switching and filter transients have settled out. The timing of the second sampling point can be selected so that there is sufficient time between the first sampling point and the second sampling point to detect a slope, but before the signal becomes too noisy.

The frequency of the energizing signal is deemed to match the resonant frequency of the sensor when the slow PLL is locked. Once the resonant frequency is determined, the measured characteristic, such as pressure and the like, is calculated using the calibration parameters associated with the sensor, which results in a difference frequency that is proportional to the measured characteristic.

In yet another aspect, the base unit can use continuous signal processing techniques instead of the sampled processing techniques discussed above. This embodiment derives continuous wave signals from the pulsed calibration signal and the pulsed sensor signal and uses the continuous wave signals to adjust the phase and frequency of the energizing signal.

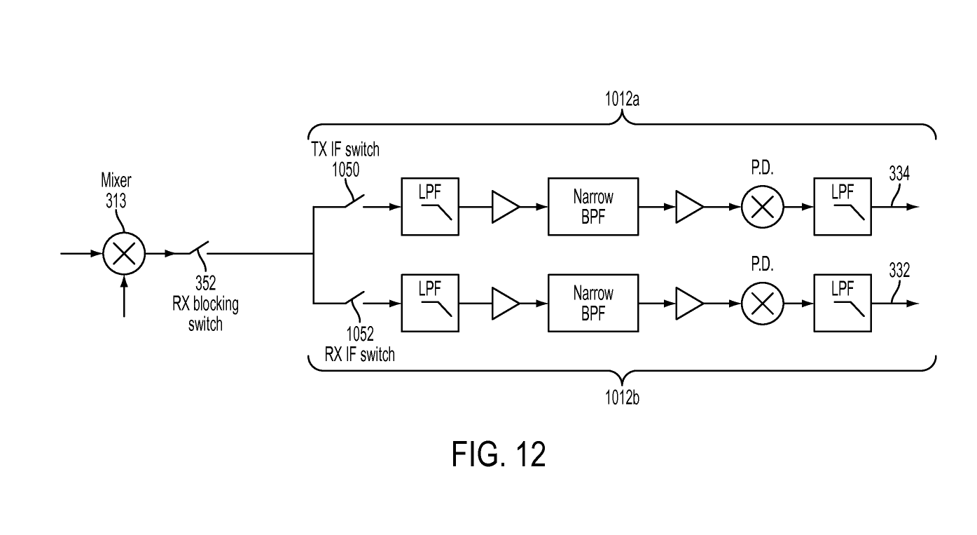

FIG. 12 illustrates a portion of the base unit for an embodiment that uses continuous signal processing. In this aspect, separate calibration 1212a and measurement sections 1212b can be used instead of the common IF section 1312 and separate sample and hold circuits 1314 and 1315 used in FIG. 11. In one aspect, after the signal passes through the receiver section 1311, the mixer, and one of the RX blocking switches, the signal is split into a pair of switches, TX IF switch 1250 and RX IF switch 1252. The FPGA controls the switches (control signals not shown) so that the TX IF switch 1250 is closed and the RX IF switch 1252 is opened during the calibration cycle and the TX IF switch is opened and the RX IF switch is closed during the measurement cycle. The calibration section 1212a and the measurement section 1212b can each include the aforementioned switch, a low pass filter, a narrow bandpass filter, amplifiers and a phase detector. The common IF section of FIG. 11 can use a bandpass filter, typically on the order of 2-3 MHz, whereas the calibration and measurements sections of FIG. 12 can use a narrow bandpass filter, typically on the order of 60-120 kHz.

In one aspect, it is contemplated that the system illustrated by FIG. 12 can use alternating calibration and measurement cycles. However, it is also contemplated that the calibration cycle and the measurement cycle can be implemented in the same pulse repetition period.

During the calibration cycle, the calibration signal which enters the receiver 1310 is processed through the receive section 1311 and the calibration section 1012a. The phase difference output from the calibration section is sent to the loop filter 1316 and the adjustment of the phase of the energizing signal proceeds as described above in connection with FIG. 11.

During the measurement cycle, the energizing signal can be blocked from the receiver during the on time of the energizing signal. During the off time of the energizing signal, the receiver is unblocked and the sensor signal is received. The coupled signal is amplified and filtered through the receive section 1311 and then transferred to the measurement section 1012b. The phase difference output from the measurement section is sent to loop filter 1320 and the adjustment of the frequency of the energizing signal proceeds as described above in connection with FIG. 11.

In one aspect, the RX blocking switches close as described above in connection with FIG. 11, but open earlier during the measurement cycle. Instead of being closed through the end of the off time of the energizing signal, the RX blocking switches open before the end of the off time. The timing of the opening of the RX blocking switches is based on the sensor characteristics and is selected so that the switches open once the sensor signal falls below the noise level. Since most of the energy from sensor signal is received within a time period of Q/fₒ, where Q is the Q of the sensor and fₒ is the center frequency of the sensor, the RX blocking switches can be opened after approximately Q/fₒ. For example, if the Q of the sensor if 40 and the fₒ is 32 MHz, then the RX blocking switches are opened after approximately 1.25 microseconds during the measurement cycle. The Q of the sensor and an approximate fₒ of the sensor are typically known and can be used to control the timing of the RX blocking switches.

In another aspect, the sampled information is used when utilizing the sample and hold techniques and the noise after the sample point(s) is ignored. However, in this continuous signal embodiment, all of the noise is seen unless other adjustments are made. Opening the RX blocking switches once the sensor signal decays below the noise level helps reduce the noise seen by the rest of the system and improves detection of the sensor signal.

The frequency spectrum of the sensor signal includes a number of spectral components that correspond to the pulse repetition frequency, including a strong component corresponding to the center frequency of the energizing signal (fₒ). The information needed to determine the resonant frequency of the sensor can be obtained by examining the phase of the spectral component that corresponds to fₒ. The measurement section isolates the spectral component at fₒ and the resulting time domain signal is a continuous wave signal.

In various aspects, the interrogation system generates an energizing signal with a random or pseudo random frame width. For example, the pulse width can be 2 microseconds for each frame, but the frame size can be pseudo randomly selected from a plurality of possible frame sizes, such as, for example and without limitation, 6.22 microseconds, 8.76 microseconds, 11.30 microseconds and 13.84 microseconds. It is contemplated that any number of frame sizes can be used, although at some point increasing the number of possible frame sizes can increase the interrogation system complexity with only incremental improvements.

In one aspect, the minimum frame sizes generally correspond to the smallest frame size that provides a sufficient receive window and typically corresponds to the pulse width. For example, and without limitation, if the pulse width is 2 microseconds, then the minimum receive window is also about 2 microseconds, which makes the minimum frame size about 4 microseconds. However, switching times and other practical considerations related to the components used may result in a slightly larger frame size. The maximum frame size is typically based on a desired average pulse repetition rate. In this example, if the average pulse repetition rate is selected as 10 microseconds, then the maximum frame size is about 14 microseconds.

If a random or pseudo random frame width is used, then the frame width can vary between the calibration cycle and the measurement cycle or a common frame width can be used for a calibration cycle and the following measurement cycle. The use of a random or pseudo random frame width helps isolate the spectral component needed to determine the resonant frequency of the sensor and relaxes the requirements of the narrow bandpass filter used in the receive section.

Optionally, the RX blocking switch 1352 can be combined with the TX IF switch 1050 and the RX IF switch 1052 and the control of the TX IF and the RX IF switches can be modified to accommodate the combination.

In another aspect, the interrogation system can be configured to minimize potential false lock problems. Typically, a false lock occurs if the interrogation system locks on a frequency that does not correspond to the resonant frequency of the sensor. In one aspect, a false lock can arise due to the pulsed nature of the system. Since the energizing signal is a pulsed signal, it includes groups of frequencies. The frequency that corresponds to a false lock is influenced by the pulse repetition frequency, the Q of the sensor, and the duty cycle of the RF burst. For example, a constant pulse repetition frequency adds spectral components to the return signal at harmonic intervals around the resonant frequency of the sensor, which can cause a false lock. In one embodiment, false locks occur at approximately 600 kHz above and below the resonant frequency of the sensor. To determine a false lock, the characteristics of the signal are examined. For example, pulse repetition frequency dithering and/or observing the slope of the baseband signal are two possible ways of determine a false lock. In one aspect where the system locks on a sideband frequency, the signal characteristics can correspond to a heartbeat or a blood pressure waveform, for example.

In another aspect, a false lock can arise due to a reflection or resonance of another object in the vicinity of the system. This type of false lock can be difficult to discern because it generally does not correspond to a heartbeat or blood pressure waveform for example. The lack of frequency modulation can be used to discriminate against this type of false lock. Changing the orientation of the magnetic loop can also affect this type of false lock because the reflected false lock is sensitive to the angle of incidence.

In yet another aspect, a false lock can arise due to switching transients caused by switching the PIN diodes and analog switches in the RF path. These transients cause damped resonances in the filters in the receive chain, which can appear similar to the sensor signal. For example, these types of false locks do not typically correspond to a heartbeat or blood pressure waveform because they are constant frequency. These types of false locks are also insensitive to orientation of the magnetic loop.

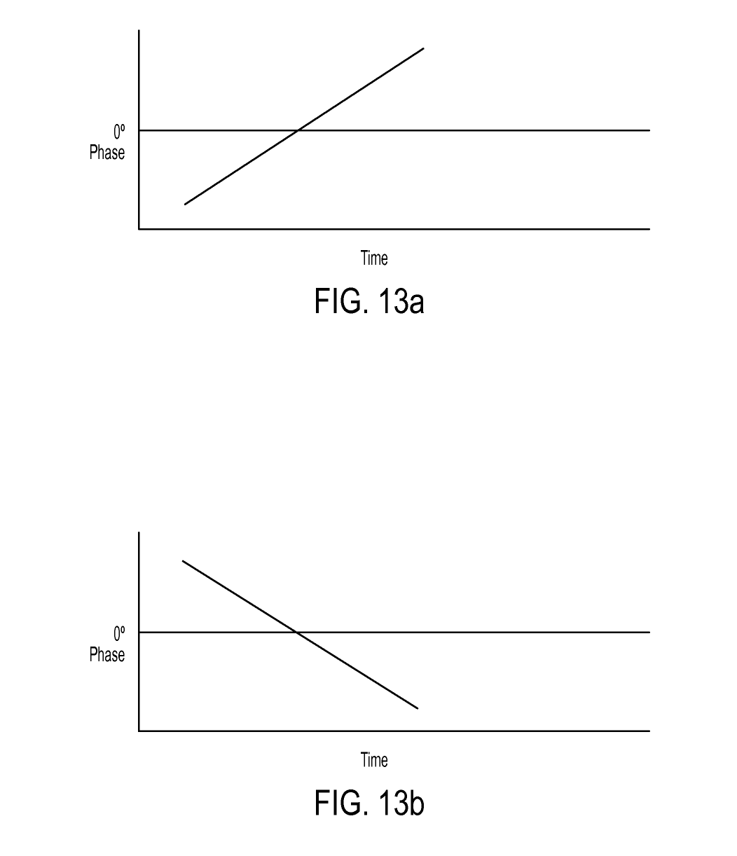

In one exemplary aspect, the interrogation system 40 can be configured to prevent the occurrence of a false lock resulting from interrogation system locking on a frequency that does not correspond to the resonant frequency of the sensor. In this aspect, to avoid the false lock, the interrogation system determines the slope of the baseband signal (the phase difference signal at point 330). In one aspect, if the slope is positive, then the lock is deemed a true lock. However, if the slope is negative, then the lock is deemed a false lock. In another embodiment, a negative slope is deemed a true lock and a positive slope is deemed a false lock. The slope is determined by looking at points before and after the phase difference signal goes to zero. The slope can be determined in a number of different ways, including but not limited to, using an analog differentiator or multiple sampling. FIGS. 13A and 13B illustrate a true lock and a false lock respectively, when a positive slope indicates a true lock.

In another aspect, if a false lock is detected, then the signal strength can be suppressed so that the signal strength appears to the processor to be below the threshold and the system continues to search for the center frequency. In other aspect, any non-zero slope can be interpreted as a false lock resulting in zero signal strength.

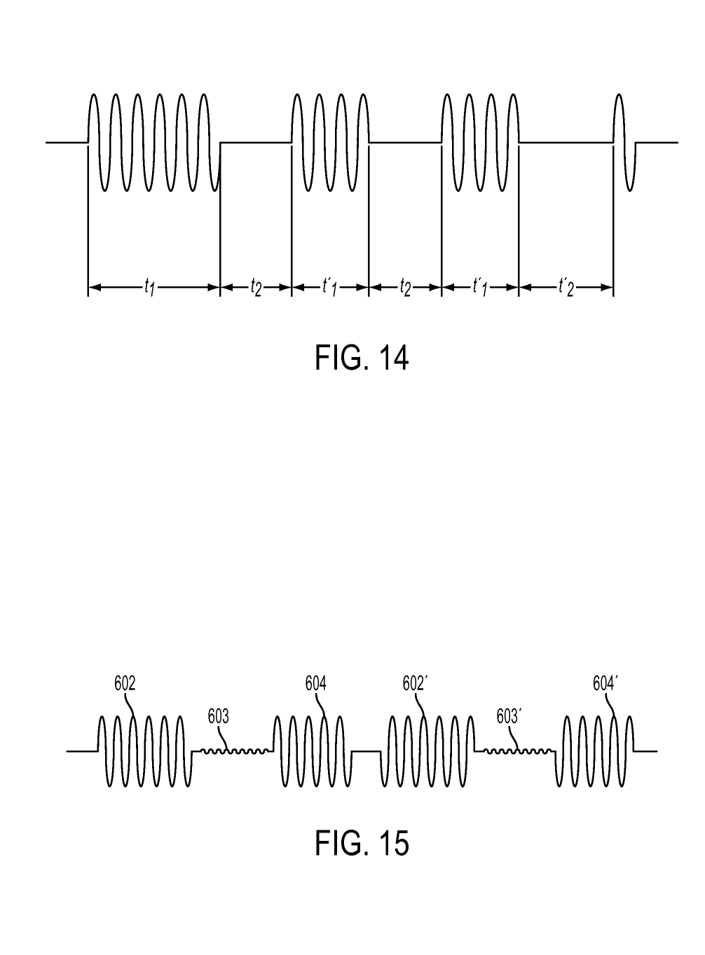

In one aspect, the interrogation system can also use frequency dithering to avoid the occurrence of a false lock resulting from interrogation system locking on a frequency that does not correspond to the resonant frequency of the sensor. In this aspect, since the spectral components associated with a constant pulse repetition frequency can cause a false lock, dithering the pulse repetition frequency helps avoid a false lock. By dithering the pulse repetition frequency, the spectral energy at the potential false lock frequencies is reduced over the averaged sampling interval. As shown in FIG. 14, the energizing signal includes an on time t1 and an off time t2. The system can vary the on time or the off time to vary the PRF (PRF=1/(t1+t2)). FIG. 14 illustrates different on times (t1, t1′) and different off times (t2, t2′). By varying the PRF, the sidebands move back and forth and the average of the sidebands is reduced. Thus, the system locks on the center frequency rather than the sidebands. The PRF can be varied between predetermined sequences of PRFs or can be varied randomly.

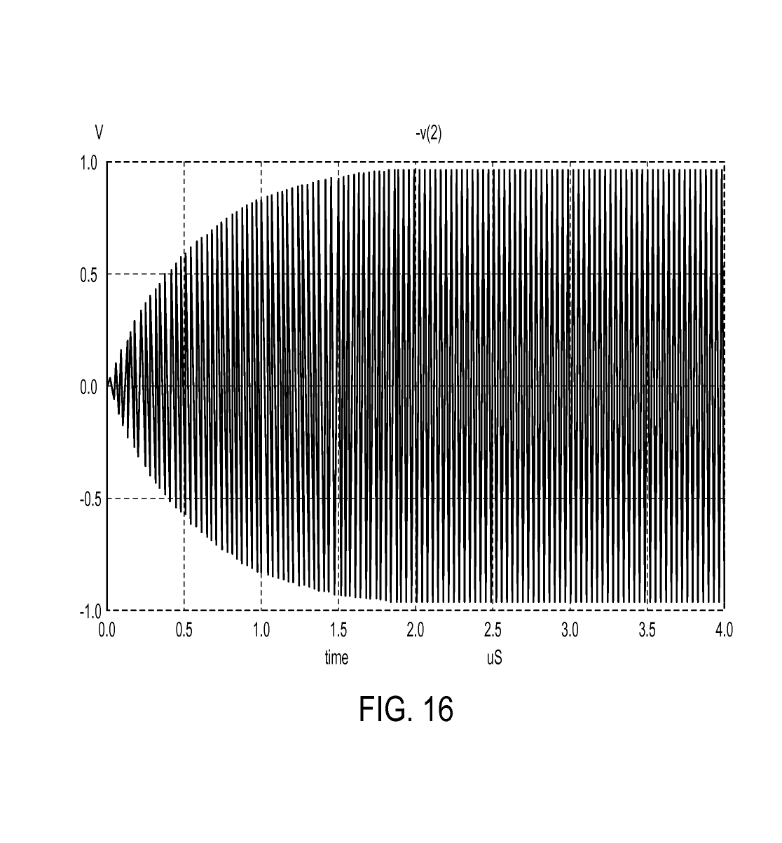

In another aspect, the coupling loop can switch between an energizing mode and a coupling mode. This switching can create transient signals, which can cause a false lock to occur. In one aspect, phase dithering is one method that can be used to reduce the switching transients. As shown in FIG. 15, the system receives a switching transient 1603 between the end of the energizing signal 1602 and the beginning of the coupled signal 1604. To minimize the transient, the phase of the energizing signal may be randomly changed. However, changing the phase of the energizing signal requires that the system redefine zero phase for the interrogation system. To redefine zero phase for the interrogation system, the phase of DDS2 is changed to match the change in phase of the energizing signal. Thus, the phase of the energizing signal 1602′ and the coupled signal 1604′ are changed, but the phase of the transient signal 1603′ is not. As the system changes phase, the average of the transient signal is reduced.

Optionally, changing the resonant frequency of the antenna as it is switched from energizing mode to coupling mode also helps to eliminate the switching transients. The coupled signal appears very quickly after the on period of the energizing signal and dissipates very quickly. In one embodiment, the invention operates in a low power environment with a passive sensor so that the magnitude of the coupled signal is small. In one exemplary aspect, the coupling loop can be tuned to a resonant frequency that is based upon the sensor parameters. Changing the capacitors or capacitor network that is connected to the coupling loop changes the resonant frequency of the antenna. In one aspect, the resonant frequency can be changed from approximately 1/10% to 2% between energizing mode and coupled mode. Additionally, in some aspect, the coupling loop is untuned.

In one aspect, the monitoring system is configured to determine pressure and other physiological data from the resonant frequency received from the implanted passive sensor. In one aspect, it is contemplated that the processing of the determined pressure and other physiological data from the resonant frequency received from the implanted passive sensor can be done in whole or in part within the interrogation system 40 or, optionally, orgation system the processing of the determined pressure and other physiological data from the resonant frequency received from the implanted passive sensor can be done in whole or in part within processors external to, and potentially remote from, the interrogation system 40.

In one aspect, the resonant frequency produced by the actuated pressure sensor is received by the interrogation system. This resonant frequency is then processed and transformed into a pressure reading that is commensurate to the actual real time pressure proximate the sensor therein the patient's vasculature. In various aspects, it is contemplated that the monitoring system can be configured to do one or more of: using a serial port to acquire the produced resonant frequency and signal strength data; using a serial port to control the interrogation system as described above; using a serial port to acquire barometric pressure from a barometric sensor proximate the patient location; handling initial setup new patient requests by taking patient demographic information, the sensor serial number, sensor calibration and sensor f₀ (for example and without limitation, the sensor information can come from a thumb drive or other similar storage medium).

The monitoring system can also be configured to do one or more of the following: displaying the signal strength from the coupled pressure sensor; initiating a frequency scan to locate the sensor within a prescribed frequency range if the signal strength is weak; calculate the pressure measured by the pressure sensor given a calibrated pressure sensor during the measurements, the software will calculate the pressure measured by the pressure sensor; handle data acquisition requests by acquiring several seconds of measured pressure data, which can include systolic, diastolic, pulse and mean pressure values for the acquisition period and which can be stored to the hard drive. The associated waveform data can also be stored to the hard drive. In an additional aspect, at the end of a recording session, the software can transmit the wave form and systolic, diastolic, pulse and mean pressure data to a data server of the monitoring system.

In various aspects, it is contemplated that the system 10 described herein can be configured in one or more configurations. Without limitation and for purposes of illustrating exemplary configurations, this specification will address two configurations, a home configuration, in which the patient and the interrogation system are co-located at a domicle or location remote from the physician and the monitoring system server, and a hospital configuration, in which the patient, the interrogation system, and the hospital system are co-located within the same hospital complex.

Operationally, the monitoring system can be programmed to effect acquisition of the resonant frequency at a rate of at least 120 Hz. In this aspect, the monitoring system can be programmed to resolve differences in sensor fₒ frequency of 50 Hz and/or of differences of 1% signal strength. The monitoring system also can be programmed to effect acquition of the barometric pressure sensor at a predetermined rate, such as, for example and without limitation, at least once every 15 minutes.

The data will be captured by the monitoring system 60 when a control to initiate data capture has been selected. In one aspect, an error message can be generated if the signal strength is 40 or less. In this case, the remaining data collection steps described below will not proceed.

It is contemplated that the data capture can record the a predetermined period of time, such as, for example, between 10-20 seconds. It is contemplated that the predetermined period of time can differ between the respective home and hospital configurations. Optionally, in the hospital configuration, after the data capture has been made, the user will be prompted and asked whether the software should keep the reading captured; the responses from the pressure dialog will be stored with the captured data; and the responses from the pressure dialog will be stored with the captured data. It is also contemplated that, irrespective of the response to the print/save query, the captured data, which can be formed of the sensor ID, date and time, can be stored to local file. In another aspect, and prior to storage, the waveform can be filtered with a 40 Hz cutoff. In another aspect, the monitoring system will save a list of frequency adjustments that were applied when computing the pressure.

After a successful implantation of the pressure sensor, a data review can be run. A screen can be provided having at least one of the following displayed elements: 1) graphical sensor pressure tracing, 2) numeric values of sensor pressure average systolic, diastolic, mean, heart rate and cardiac output, 3) swan pressure user entered scalar systolic, diastolic and mean displayed as horizontal lines on the graph, 4) numeric values of swan pressure user entered scalar systolic, diastolic and mean, 5) reading date and time, and/or 6) sensor signal strength displayed. In one aspect, if the sensor diastolic pressure is greater than or equal to 0 then the monitoring system will display the data relative to the Y axis scaled from 0 to 100 mmHg and if the sensor diastolic pressure is less than 0 then the software will display the data relative to the Y axis scaled from −20 to 80 mmHg.

In one further aspect, it is contemplated that the user will be able to modify the swan pressure systolic, diastolic and mean values. In this aspect, by selecting a sensor adjust control, a dialog indicating the average pressure difference between the sensor and swan mean pressure values for all valid readings with valid swan values can be accessed and the difference between the average of the valid sensor and swan mean pressure values shown can be increased or decreased by 1 mmHg.

In another aspect, the monitoring system can be configured to display the determined physiological values as dashed lines if the signal strength received by the interrogation system is less than a predetermined value, such as, for example and without limitation, 40, to ensure that the user is notified that the data is potentially unreliable. In a further display aspect, the waveform can be scaled such that the Y axis ranges 100 mmHg. For example, if the pressure is calibrated, the waveform can be scaled 0 to 100 mmHg. Optionally, the monitoring system can optionally display axis scales ranging between: 0-25, 0-50, 0-75, 0-100, −25-25, −25-50, −25-75, and −25-100 mmHg.

In another aspect, the monitoring system will apply any adjustments to sensed resonant frequency prior to computing pressure. In this aspect, the adjustments at standard frequencies for a given sensor 20 are established at the time of manufacturing the sensor and are stored in a configuration file in the monitoring system. It is contemplate that the monitoring system will linearly interpolate the necessary adjustment when the determined resonant frequency is between the standard frequencies. In one exemplary aspect, the absolute pressure value can be calculated from the acquired resonant frequency value with adjustments determined by the following formula:

P_{abs}=sf2*f̂2+sf1*f+C offset,

where f is the current resonant frequency value, sf2 and sf1 are the sensor calibration factors, which are stored in the monitoring system for the particular sensor upon setup, and C offset is the calibrated offset of the respective sensor, which is determined upon implantation. Typically, the displayed pressure data is modified by barometric pressure to compensate for the fact that the respective pressure sensor 20 measures absolute pressure and will therefore display pressures at higher atmospheric pressures. In this step, the P_{gauge} will be determined by subtracting the current barometric pressure, which can be adjusted for from the determined absolute pressure.

In a further aspect, the monitoring system can examine the determined pressure/time waveform for the purpose of identifying blood pressure beats. In this aspect, the system can be configured to detect detected blood pressure beats that are, for example and without limitation, at least 4 mmHg in amplitude and less than or equal to 100 mmHg in amplitude, and have a rise time of systolic at least 33 ms and less than or equal to 400 ms. If the monitoring system is able to detect the blood pressure beats, the monitoring system can be configured to calculate the patient's respective systolic, diastolic and mean values for the length of time the waveform is displayed on the screen. Further, if the software is able to detect the blood pressure beats, the monitoring system can be configured to calculate the heart rate by using the diastolic to diastolic time of the blood pressure beats for the time period displayed on the waveform graph.

Optionally, if the monitoring system is able to detect the blood pressure beats, the monitoring system can calculate the cardiac output using the blood pressure beats for the waveform time length on the screen. In one aspect, the cardiac output will be calculated by using the following formula:

CO(L/min)=VTI*HR(BPM)*Aortic Area (sq. cm)/1000

where VTI is equal to 100 times the time integral from start to end of systole of the square root of relative pressure over 4, where the relative CardioMEMS pressure is the CardioMEMS pressure relative to a line from start to end systole. In one aspect, the monitoring system can define asystole as the shorter value of either the time window length or 10 seconds. For asystole, the monitoring system can calculate the systolic, diastolic and mean pressure by the maximum, minimum and mean (respectively) of the pressure waveform on the screen. Optionally, prior to calculating the systolic, diastolic and mean pressure, the monitoring system can filter the waveform with cutoff, such as an exemplary 40 Hz cutoff. In another aspect, it is contemplated that the heart rate and cardiac output will not be displayed if the software is not able to detect the blood pressure beats and further, cardiac output will not be shown on the display screen if the system does not have cardiac output calibrated.

In a further aspect, the display screen can have a bar graph indicator that is configured to show the relative signal strength of the pressure sensor. In one aspect, the displayed signal strength can vary from between about 0 to 99%.

In another aspect, the monitoring system can compute the spectrum magnitude of the received resonant frequency. A pulsatile signal is defined as one with the frequency content below 13 Hz is at least two times the frequency content above 13 Hz. However, it is contemplated that the noted frequency criteria for pulsatile must be present for a predetermined time period, such as, for example and without limitation, 10 seconds, for the monitoring system to declare the signal as potentially pulsatile. Optionally, the display can provide graph indicators with the following colors: blue, which indicates a signal strength greater than 40 and the frequency is non-pulsatile; green, which indicates a signal strength greater than 60 and the frequency is pulsatile; yellow, which indicates a signal strength greater than or equal to 60 and the frequency is pulsatile; and white, which indicates a signal strength less than or equal to 40.

In operation, a patient will position themselves relative to the antenna of the interrogation system and initiate the measurement process. Once the pressure measurement process is initiated, a log file entry can be generated and a cancel button can be displayed to stop the current measurement. If the cancel button is pressed, a log message of the action can be generated or otherwise recorded. Optionally, once the pressure measurement sequence initiated, the monitoring system can instruct the patient to position themselves relative to the antenna in the correct location for measurements.

Typically, the monitoring system will wait a predetermined period of time from when the measurement has started before indicating the reception of a good or bad signal. This predetermined period of time can range between 10-15 seconds, preferably between about 15-20 seconds, and most preferably about 18 seconds. In a further aspect, the monitoring system can graphically display the current signal strength being detected to the patient to assist the patient in locating the correct position for pressure measurements. While the signal strength is below the threshold for recording, the monitoring system can be configured to play a tone every 2 seconds that can be proportional to the signal strength such that 0-100% signal strength maps to 100-500 Hz. Next, when the monitoring system detects pulsatile signal with an adequate signal strength greater than 60, it can be configured to instruct the patient to hold their position of adequate signal strength and to press the take reading button. Optionally, the patient can also receive an audible indication that they are in the correct position.

When the patient is in position and the monitoring system is receiving a pressure signal with the correct characteristics to make a measurement then a reading will begin. Once the reading has begun, then the monitoring system can play a musical sequence to indicate that the measurement is proceeding. Once the patient is in position, the software will ensure that the signal is pulsatile with a signal strength is greater than the minimum signal strength then a reading will be taken. Once the pressure measurement has been successfully captured, a log message can be generated and it can be subsequently analyzed and scored as using the HF Reading Scoring Algorithm described herein below. If the first reading data score is higher than a preset minimum, a message will be shown to the user to effect a restart of the data acquisition process described above. In this aspect, it is contemplated that, once the user confirms the message to retake the reading, the monitoring system will not immediately transmit but will instead transition to the start button so that the user may initiate the reading. If the second reading data score is higher than the present minimum, no message will be sent to the user and no further reading will be taken at than time. It is contemplated that data received in the second reading can be forwarded to the server of the monitoring system even in the instance when the data is scored as “bad” data. If the score of the reading is lower than the present minimum, the reading data can be stored and the user can be presented with a display and, optionally, an accompanying audible alert that the reading is complete.

Subsequent to a successful reading, the monitoring system incorporated therein the interrogation system will cue the reading data for transmission to the server in the monitoring system. The reading data transmitted can include one or more of the following: the sensor serial number, the date/time of when reading was taken, the patient identifier, the determined systolic, diastolic and mean pressures, average frequency of reading, average and minimum signal strength of reading, point by point pressure waveform samples, cardiac output, heart rate, point by point signal strength waveform, interrogation system serial number, implant calibration intercept of pressure, implant calibration area of cardiac output, version of software on the interrogation system, sensor slope 1 and slope 2, atmospheric pressure, and sensor type, which can indicates where the respective sensor is located within the patient. Upon a valid connection, the monitoring system can be configured to synchronize the configuration data with the server using the following steps. 1) notify the server of md5 of file, 2) if the server responds to send the file to the server then the file will be sent, 3) if the server responds to receive a file from the server then the file will be received, and 4) if the server responds that the file is up to date then no further action will be taken. After, the reading data is transferred or all connection attempts have been made, the monitoring system can be configured to instruct the interrogation system to power down.

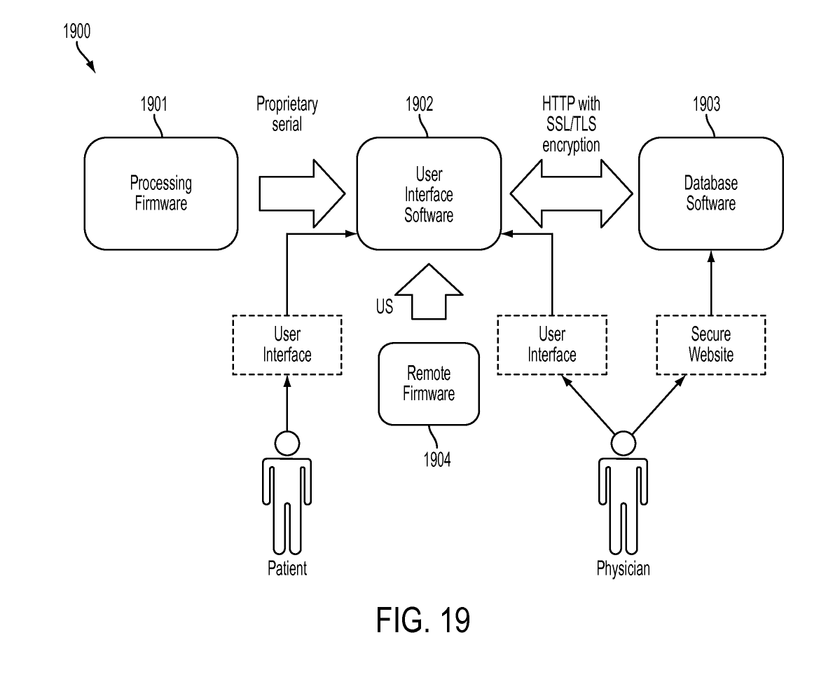

In one exemplary aspect, the monitoring system 60 can include all processing code required for operation of the system, from acquisition of the sensor signal by the interrogator 40 to the transmission of the sensor signal to a database, to the display of readings on an associated website. The monitoring system 1900 can be separated into four distinct sub-systems, and integrated as depicted in FIG. 19. The processing firmware 1901 can be the firmware for the sub-components of the interrogator 40 required to operate the various aspects of the interrogator, for example and without limitation, the RF transmitter, the phase-locked loops, and the receiver circuitry. The user interface software 1902 can be software located on a processor within the interrogator 40 and programmed to process, display, store, transmit and allow for user interaction with the sensor signal data and other data stored on the interrogator such as, for example and without limitation, the atmospheric pressure, the sensor serial number, the sensor calibration data, and the like. The database software 1903 comprises software located on a server that is programmed to retrieve remote data from the interrogator, store received and processed data and allow a user to access the data. The remote firmware 1904 can be the firmware programmed to manage the communication of a hand-held component and the database software.

In another exemplary aspect, a home configuration of the system comprises an interrogation system that can house signal generation and processing components and can comprise RF transmit and receive and processing circuitry, a single board computer, and interface ports. The RF and processing circuitry can be operable to generate bursts of RF energy to powers the sensor, process the return signal from the sensor, and transmit pressure information to the single board computer. This circuitry may also contain an atmospheric pressure sensor operable to provide data to compensate for changes in altitude and atmospheric pressure. The single board computer can be operable to process and store data for the interrogator 40. A USB storage device (thumb drive) connected through the USB port can be used to provide information associated with the sensor such as, for example and without limitation, a sensor serial number or sensor calibration data. In the absence of the USB storage device, a user can manually enter the calibration coefficients, which can be provided on a patient implant card unique to the sensor or affixed or printed on the sensor packaging. Once entered, this information can be transmitted to the single board computer. This data can include, e.g., a waveform graph, the calibration coefficients, and averaged readings, such as diastolic, systolic, mean, and pulse pressure. A USB port can be provided that is operable to facilitate the transfer of data and also enable software upgrades. The interrogator can have mechanisms for transmitting patient data either through the Plain Old Telephone System (POTS), through wireless (GSM) communication, through the internet or other communication means known in the art.

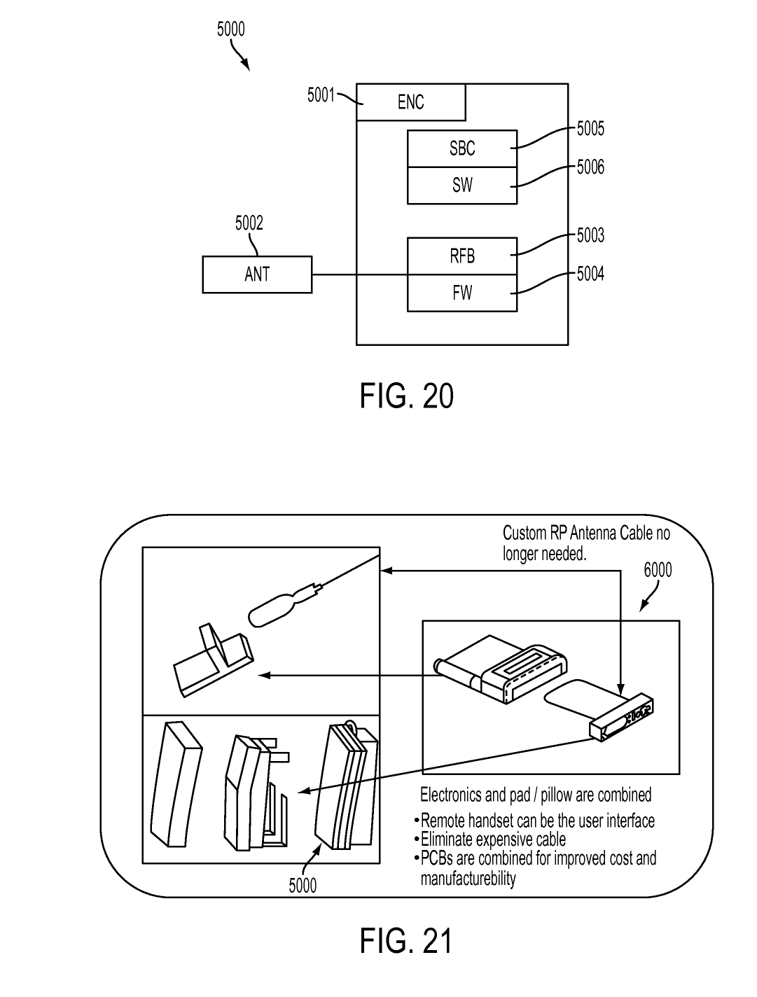

In another aspect and as illustrated in FIG. 20, a system architecture for an interrogator main unit 5000 can include an enclosure 5001, an antenna 5002, an RF processing board 5003, firmware 5004, a single board computer 5005, and software 5006. The enclosure 5001 can set the form factor of the interrogator, can incorporate the user interface touch sere and can provide mechanical protection as well as EMI/EMC protection through internal shielding. The size of the enclosure can be based on intended use environment during measurement. The antenna 5002 can wirelessly provide power to the sensor and can receive a return signal from the sensor, can connect to the interrogator via a cable, and can be both flexible and housed within a soft cushion or pillow. The RE processing board 5003 comprises the RF circuitry configured to interact with the sensor and more particularly comprises the RF transmitting circuitry, the RF receiver circuitry, the microprocessor operable to send the frequency data to the single board computer and the POTS and/or GSM modem circuitry. The firmware 5004 for the FPGA and microprocessor(s) is located on the RF processing board and can be configured to communicate frequency data to the single board computer, provide firmware to interface a remote handheld component to the microprocessor and provides the phase-lock loop control for the RE transmitter and receiver as well as the timing for the sampling and isolation switching within the receiver. The single board computer 5005 can comprise the computer circuit board, which can contain the hardware required for user interface I/O USB, touch screen, etc. . . . ), the microprocessor used for the user I/O and the memory device for software and data. The software 5006 can comprise, for example and without limitation Linux, C based application software, user interface software that controls the user I/O, software operable to convert frequency data from the RE processing board to pressure data, and the like. In one exemplary embodiment of the interrogator, the form factor of the interrogator can be configured such that an interrogator main unit can sit on a nightstand or similarly-sized table while the antenna comprising the coupling loop(s) is incorporated into a pad or pillow and an RF cable connects the antenna to the interrogator main unit.

In another aspect illustrated in FIG. 21, an alternative configuration for a home system is depicted. Here, the interrogator main unit and the antenna connected via an RF cable in the system depicted in FIG. 20 are consolidated into an a single interrogator unit 6000 eliminating the RF cable. In this configuration, a touch screen user interface may be incorporated into a small handset associated with the system. It is contemplated that the touch screen can be integrated into the single interrogator unit or can be incorporated into a small handset which can be stored attached to the unit. The single interrogator unit 6000 can optimize the interrogator design to reduce the volume of the enclosure required for the circuit boards and can be integrated into the antenna pillow. The antenna cable is no longer needed, eliminating one hardware component, and the GSM modem circuitry can be connected via USB. Here, firmware can be utilized to interface a remote device to the microprocessor or use software via a touch screen interface that is directly connected to the interrogator unit 6000.

In other aspects, both the interrogator main unit 5000 and the single interrogator unit 6000 have the following functional and performance characteristics: The accuracy of the unit can be about +/−2 mmHg at baseline pressure against standard and about +/−3 mmHg of difference when measured between pressure and baseline. The noise of the unit can have a resolution of about 1 mmHg. The detection distance of the unit can be about 6 inches and achieve a detection distance of about 4 inches at an angle of about 60 degrees rotation relative to the sensor. Additionally or alternatively, the unit can detect the sensor in the pulmonary artery of a patient. In other aspects, the unit can be configured to have repeatability of measurements within +/−1 mmHg and can be configured to lock onto a sensor from 500 kHz (or 25 mmHg assuming 20 kHz per mmHg) or have a means for the user to force a search window. In other aspects, the unit can be configured to display a sample rate of about or greater than 120 Hz and, in light of the present disclosure, one skilled in the art will appreciate that this is based on the Nyquist Criteria with the assumption that the frequency components of the pressure waveform are from about 0 to about 60 Hz. Additionally, the unit must comprise a means for calibration which meets the accuracy requirements of the unit and the unit can comprise a means of entering the mean value of an alternative measurement such as a swan-ganz pressure catheter. In other aspects, the unit can be configured to measure barometric pressure within about +/−2 mmHg at baseline pressure against standard and about +/−3 mmHg of difference when measured between pressure and baseline. In yet other aspects, the user interface of the unit can be configured to indicate when sensor signal strength meets a low threshold value and that threshold value can be about 40%. In other aspects, the unit can be configured to continue searching for a stronger sensor signal when the sensor signal strength is below the threshold value. In other aspects, the unit can be configured to have an operational frequency range of from about 30 to about 37.5 MHz. In even other aspects, the unit can further be configured to measure from about 0 to about 100 mmHg at altitudes between about 0 and about 5,280 feet.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is therefore understood that the invention is not limited to the specific embodiments disclosed herein, and that many modifications and other embodiments of the invention are intended to be included within the scope of the invention. Moreover, although specific terms are employed herein, they are used only in a generic and descriptive sense, and not for the purposes of limiting the described invention.

Various publications are referenced in this document. These publications in their entireties are hereby incorporated by reference into this application in order to more fully describe the state of the art to which the disclosed system and method pertains. The references disclosed are also individually and specifically incorporated by reference herein for the material contained in them that is discussed in the sentence in which the reference is relied upon.

What is claimed is:

1. A method for treating congestive heart failure in a patient, comprising:
implanting at least one pressure sensor in a desired location within the patient;
providing an ex-vivo interrogation system and monitoring system that can be configured to optionally affect at least one of: selectively energizing the at one pressure sensor, receiving a return or output signal from the at one pressure sensor, processing the return signal, and displaying processed data derived from the at least one pressure sensor to a physician, wherein the processed data comprises at least one of a sensor serial number, a date/time of when reading was taken, a patient identifier, determined systolic, diastolic and mean pressures, average frequency of reading, average and minimum signal strength of reading, point by point pressure waveform samples, cardiac output, heart rate, point by point signal strength waveform, interrogation system serial number, implant calibration intercept of pressure, implant calibration area of cardiac output, version of software on the interrogation system, sensor slope 1 and slope 2, atmospheric pressure, and sensor type.

2. The method of claim 1, further comprising deriving diagnostic and treatment information from the processed data.

3. The method of claim 2, further comprising sending diagnostic and treatment information to the patient.

4. The method of claim 3, further comprising allowing the patient to continue or modify prescribed medications to achieve optimal therapeutic doses generally without direct intervention of the physician.

5. A system for treating congestive heart failure in a patient, comprising:
at least one pressure sensor comprising a resonant circuit that is operable to generate a sensor signal that is indicative of a pressure when positioned in a desired location within a patient and energized;
an ex-vivo interrogation system comprising a base unit having a receiver, wherein the base unit is operably connected to a coupling loop operable to selectively energize the at least one pressure sensor and couple the sensor signal of the sensor into the receiver, wherein the interrogation system is operable to communicate the sensor signal to a location outside of the patient;
an ex-vivo monitoring system comprising at least one processor operatively coupled to the ex-vivo interrogation system, wherein the monitoring system is programmed to convert the sensor signal to a pressure signal and determine and output at least one type of processed data based on the at least one pressure sensor, the ex-vivo monitoring system and the determined pressure signal.

6. The system of claim 5, wherein the interrogation system further comprises a user interface.

7. The system of claim 6, wherein the user interface comprises a display device.

8. The system of claim 6, wherein the user interface comprises an input device.

9. The system of claim 5, wherein the coupling loop further comprises a transmit loop and a receive loop.

10. The system of claim 5, wherein the monitoring system and the interrogation system are at least partially coextant.

11. The system of claim 5, wherein the at least one pressure sensor further comprises a monolithic housing.

12. The system of claim 5, wherein the sensor signal comprises a resonant frequency of the resonant circuit.

13. The system of claim 5, wherein the pressure sensor and the interrogation system are co-located.

14. The system of claim 13, wherein the monitoring system is remote from the sensor and interrogation system.

15. The system of claim 13, wherein the monitoring system is co-located with the sensor and interrogation system.

16. The system of claim 5, wherein processed data is selected from the group comprising: comprises at least one of a sensor serial number, a date/time of when reading was taken, a patient identifier, determined systolic, diastolic and mean pressures, average frequency of reading, average and minimum signal strength of reading, point by point pressure waveform samples, cardiac output, heart rate, point by point signal strength waveform, interrogation system serial number, implant calibration intercept of pressure, implant calibration area of cardiac output, version of software on the interrogation system, sensor slope 1 and slope 2, atmospheric pressure, and sensor type.
